(19) United States
(12) Patent Application Publication
(10) Pub. No.: US 2023/0057531 A1
(43) Pub. Date: Feb. 23, 2023

(54) MOBILE DEVICE STANDS FOR AT-HOME DIAGNOSTICS AND HEALTHCARE

(71) Applicant: EMED LABS, LLC, Miami, FL (US)

(72) Inventors: Zachary Carl Nienstedt, Wilton Manors, FL (US); Colman Thomas Bryant, Fort Lauderdale, FL (US); Binsen Josuhe Mejia, Palmetto Bay, FL (US); Marco Magistri, Miami, FL (US); Christopher Richard Williams, Miami, FL (US); Sam Miller, Hollywood, FL (US)

(21) Appl. No.: 17/819,900

(22) Filed: Aug. 15, 2022

Related U.S. Application Data
(60) Provisional application No. 63/260,504, filed on Aug. 23, 2021.
(60) Provisional application No. 63/260,349, filed on Aug. 17, 2021.
(60) Provisional application No. 63/261,883, filed on Sep. 30, 2021.

(51) Int. Cl. G16H 40/67

(52) U.S. Cl. CPC G16H 40/67

(57) ABSTRACT

This disclosure relates to systems and methods for remote diagnostic medical testing. In some embodiments, a remote testing method can include receiving a first image frame, determining a first area of the first image frame, identifying a first feature in the first area, and providing an indication that the first feature is in the first area of the first image frame, and continuing a remote diagnostic testing session. Some embodiments relate to test kits and contents there. In some embodiments, a test kit can include a stand configured to hold a user device during a testing session. In some embodiments, a test kit can include packing configured to hold test kit components in a particular configuration. In some embodiments, a test kit can include a reference card.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

This application claims the benefit under 35 U.S.C. § 119(e) to U.S. Provisional Application No. 63/260,504, filed Aug. 23, 2021, U.S. Provisional Application No. 63/260,349, filed Aug. 17, 2021, entitled “SYSTEMS, METHODS, AND DEVICES FOR DIAGNOSTIC TEST KITS,” and 63/261,883, filed Sep. 30, 2021, entitled “POSITIONING GUIDANCE FOR REMOTE MEDICAL DIAGNOSTIC TESTING,” and the entirety of these applications is hereby incorporated by reference herein for all purposes.

### BACKGROUND

### Field

Some embodiments of the present disclosure are directed to systems and methods for conducting remote health testing and diagnostics. In particular, some embodiments of the present disclosure are directed to mobile device stands for use with remote health testing and diagnostics.

### Description of Related Art

Use of telehealth to deliver health care services has grown consistently over the last several decades and has experienced very rapid growth in the last several years. Telehealth can include the distribution of health-related services and information via electronic information and telecommunication technologies. Telehealth can allow for long-distance patient and health provider contact, care, advice, reminders, education, intervention, monitoring, and remote admissions. Often, telehealth can involve the use of a user or patient's personal device, such as a smartphone, tablet, laptop, personal computer, or other type of personal device. For example, the user or patient can interact with a remotely located medical care provider using live video and/or audio through the personal device.

Remote or at-home health care testing and diagnostics can solve or alleviate some problems associated with in-person testing. For example, health insurance may not be required, travel to a testing site is avoided, and tests can be completed at a patient's convenience. However, at-home testing introduces various additional logistical and technical issues, such as guaranteeing timely test delivery to a patient's home, providing test delivery from a patient to an appropriate lab, ensuring test verification and integrity, providing test result reporting to appropriate authorities and medical providers, and connecting patients with medical providers, who are needed to provide guidance and/or oversight of the testing procedures remotely.

### SUMMARY

Health testing and diagnostics platforms are provided herein that can facilitate proctored or video-based at-home or remote healthcare testing and diagnostics. For example, users performing at home testing may be guided or assisted by proctors that are available over a communication network using, for example, live video on via the users' devices.

User devices can provide an important tool for enabling affordable remote and at-home healthcare. The user experience of remote or at-home healthcare can depend on how the user device is used, physically and digitally. For example, the physical side of this can include how the device is positioned and how the user interacts with it. Device stands can be used, which can, in some examples, be integrated into the product, to provide the proper positioning of the user device. In some embodiments, systems and methods herein may help to ensure adequate image quality. This can lead to improved accuracy of test results, a more pleasant user experience, and other benefits.

In some aspects, the techniques described herein relate to a method for remote diagnostic testing including: receiving, by a computer system from a user device, a first image frame; determining, by the computer system, a first area of the first image frame; identifying, by the computer system, a first feature in the first area; and providing, by the computer system to a user via the user device, an indication that the first feature is in the first area of the first image frame; and continuing, by the computer system, a remote diagnostic testing session.

In some aspects, the techniques described herein relate to a method, further including: receiving, by the computer system from the user device, a second image frame; identifying, by the computer system, a second area of the second image frame; determining, by the computer system, that the first feature is not within the second area of the second image frame; providing, by the computer system to the user via the user device, an indication that the first feature is not in the second area of the second image frame; and pausing, by the computer system, the remote diagnostic testing session.

In some aspects, the techniques described herein relate to a method, further including: receiving, by the computer system form the user device, a third image frame; identifying, by the computer system, a third area of the third image frame; determining, by the computer system, that the first feature is within the third area of the third image frame; providing, by the computer system to the user via the user device, an indication that the first feature is within the third area of the third image frame; and resuming, by the computer system, the remote diagnostic testing session.

In some aspects, the techniques described herein relate to a method, wherein the second area is the same as the first area and the third area is the same as the second area.

In some aspects, the techniques described herein relate to a method, wherein the first feature includes a test kit, a swab, a test strip, a reagent bottle, or a reference card.

In some aspects, the techniques described herein relate to a method, wherein the first feature includes a reference card.

In some aspects, the techniques described herein relate to a method, wherein the reference card includes a unique identifier of a test.

In some aspects, the techniques described herein relate to a method, further including: detecting, by the computer system, one or more fiducials in the first image frame; and adjusting an alignment of the first image frame, wherein adjusted the alignment includes adjust one or more of skew and keystone.

In some aspects, the techniques described herein relate to a method, further including: identifying, by the computer system, a detection threshold calibration area; determining, by the computer system, a first color of a first region of the detection threshold calibration area; determining, by the computer system, a second color of a second region of the detection threshold calibration area; determining, by the computer system, if a difference between the first color and the second color is greater than or equal to a minimum difference value; and if the difference is greater than or equal to the minimum difference value, continuing, by the computer system, the remote diagnostic testing session; otherwise, providing, by the computer system via the user device, an indication to the user that the difference is less than the minimum difference value.

In some aspects, the techniques described herein relate to a method, further including: identifying, by the computer system, a color calibration area; extracting, by the computer system, a first color value from a first portion of the color calibration area; and adjusting, by the computer system, the first image frame based on a difference between the first color value and a first reference color value.

In some aspects, the techniques described herein relate to a method, further including: extracting, by the computer system, a second color value from a second portion of the color calibration area; and adjusting, by the computer system, the first image frame based on a difference between the second color value and a second reference color value.

In some aspects, the techniques described herein relate to a method, further including: determining, by the computer system based on the unique identifier, an expiration date of a test kit associated with the reference card; and validating, by the computer system, that the test kit is not expired.

In some aspects, the techniques described herein relate to a method, further including: querying, by the computer system based on the unique identifier, a database; and receiving, by the computer system from the database, information about the test, the information including one or more of reference card feature locations, test strip interpretation information, test strip line locations, and testing procedures.

In some aspects, the techniques described herein relate to a method, further including: determining, by the computer system, a sharpness of the first image frame.

In some aspects, the techniques described herein relate to a system for remote diagnostic testing including: a non-transitory computer-readable medium with instructions encoded thereon; and one or more processors configured to execute the instructions to cause the system to: receive, by a computer system from a user device, a first image frame; determine a first area of the first image frame; identify a first feature in the first area; and provide, to a user via the user device, an indication that the first feature is in the first area of the first image frame; and continue a remote diagnostic testing session.

In some aspects, the techniques described herein relate to a system, wherein the instructions, when executed by the one or more processors, further cause the system to: receive, by the computer system from the user device, a second image frame; identify a second area of the second image frame; determine that the first feature is not within the second area of the second image frame; provide, to the user via the user device, an indication that the first feature is not in the second area of the second image frame; and pause the remote diagnostic testing session.

In some aspects, the techniques described herein relate to a system, wherein the instructions, when executed by the one or more processors, further cause the system to: receive, by the computer system form the user device, a third image frame; identify a third area of the third image frame; determine that the first feature is within the third area of the third image frame; provide, to the user via the user device, an indication that the first feature is within the third area of the third image frame; and resume the remote diagnostic testing session.

In some aspects, the techniques described herein relate to a system, wherein the second area is the same as the first area and the third area is the same as the second area.

In some aspects, the techniques described herein relate to a system, wherein the first feature includes a test kit, a swab, a test strip, a reagent bottle, or a reference card.

In some aspects, the techniques described herein relate to a system, wherein the first feature includes a reference card.

In some aspects, the techniques described herein relate to a system, wherein the reference card includes a unique identifier of a test.

In some aspects, the techniques described herein relate to a system, wherein the instructions, when executed by the one or more processors, further cause the system to: detect one or more fiducials in the first image frame; and adjust an alignment of the first image frame, wherein adjusted the alignment includes adjust one or more of skew and keystone.

In some aspects, the techniques described herein relate to a system, wherein the instructions, when executed by the one or more processors, further cause the system to: identify a detection threshold calibration area; determine a first color of a first region of the detection threshold calibration area; determine a second color of a second region of the detection threshold calibration area; determine if a difference between the first color and the second color is greater than or equal to a minimum difference value; and if the difference is greater than or equal to the minimum difference value, continue the remote diagnostic testing session; otherwise, provide, via the user device, an indication to the user that the difference is less than the minimum difference value.

In some aspects, the techniques described herein relate to a system, wherein the instructions, when executed by the one or more processors, further cause the system to: identify a color calibration area; extract a first color value from a first portion of the color calibration area; and adjust the first image frame based on a difference between the first color value and a first reference color value.

In some aspects, the techniques described herein relate to a system, wherein the instructions, when executed by the one or more processors, further cause the system to: extract a second color value from a second portion of the color calibration area; and adjust the first image frame based on the difference between the second color value and a second reference color value.

In some aspects, the techniques described herein relate to a system, wherein the instructions, when executed by the one or more processors, further cause the system to: determine, by the computer system based on the unique identifier, an expiration date of a test kit associated with the reference card; and validate that the test kit is not expired.

In some aspects, the techniques described herein relate to a system, wherein the instructions, when executed by the one or more processors, further cause the system to: query, by the computer system based on the unique identifier, a database; and receive, by the computer system from the database, information about the test, the information including one or more of reference card feature locations, test strip interpretation information, test strip line locations, and testing procedures.

In some aspects, the techniques described herein relate to a system wherein the instructions, when executed by the one or more processors, further cause the system to determine a sharpness of the first image frame.

For purposes of this summary, certain aspects, advantages, and novel features of the invention are described herein. It is to be understood that not necessarily all such advantages may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein.

All of these embodiments are intended to be within the scope of the invention herein disclosed. These and other embodiments will become readily apparent to those skilled in the art from the following detailed description having reference to the attached figures, the invention not being limited to any particular disclosed embodiment(s).

### BRIEF DESCRIPTION OF THE DRAWINGS

The drawings are provided to illustrate example embodiments and are not intended to limit the scope of the disclosure. A better understanding of the systems and methods described herein will be appreciated upon reference to the following description in conjunction with the accompanying drawings, wherein:

FIGS. 1A-1D are illustrations of a user device including a display and camera in various time-sequential stages of a remotely administered medical test according to some embodiments.

FIG. 2 is an illustration of a user device including a display showing two camera views according to some embodiments.

FIG. 3 illustrates a user taking a remote health or diagnostic test using a test kit and a user device, such as a mobile phone, according to some embodiments as described herein.

FIG. 4 illustrates a field of view associated with a camera of a user device, such as a mobile phone, during administration of a remote health of diagnostic test, according to some embodiments described herein.

FIG. 5 is a table summarizing testing of different user device positions during administration of a remote health or diagnostic test, according to some embodiments described herein.

FIG. 6 illustrates six different positions for a user device during administration of a remote health or diagnostic test, according to some embodiments described herein.

FIGS. 7A and 7B illustrate embodiments of a test kit and various features that can be included therein.

FIGS. 8A and 8B illustrate embodiments of a box and insert that can be included in a test kit as well as various features that can be included therein.

FIG. 9 illustrates an embodiment of a test kit in an example testing configuration.

FIGS. 10A-10E illustrate an example device stand that can be integrated into a remote health or diagnostic test kit according to some embodiments described herein.

FIGS. 11A-D illustrate various views of an example device stand that can be integrated into a remote health or diagnostic test kit according to some embodiments described herein.

FIGS. 12A and 12B illustrate various views of an example device stand that can be integrated into a remote health or diagnostic test kit according to some embodiments described herein.

FIGS. 13A and 13B illustrate various views of an example device stand that can be integrated into a remote health or diagnostic test kit according to some embodiments described herein.

FIGS. 14A and 14B illustrate various views of a foldable example device stand that can be used with, for example, a remote health or diagnostic test kit according to some embodiments described herein.

FIGS. 15A-15C illustrate various views of another foldable example device stand that can be used with, for example, a remote health or diagnostic test kit according to some embodiments described herein.

FIGS. 16A and 16B illustrate examples of device stands according to some embodiments described herein.

FIGS. 17A-17C illustrate various views of an example device stand that can be integrated into a remote health or diagnostic test kit according to some embodiments described herein.

FIGS. 18A and 18B illustrate various views of an example device stand that can be integrated into a remote health or diagnostic test kit according to some embodiments described herein.

FIG. 19 illustrates an embodiment of a reference card for a test kit and various features that can be included thereon.

FIGS. 20A and 20B illustrate embodiments of swabs for a test kit and various features that can be included thereon.

FIG. 21 illustrates an embodiment of a test strip for a test kit included various features that can be included thereon.

FIG. 22 illustrates an embodiment of an example test flow or process.

FIGS. 23A and 23B illustrate example embodiments of various components that can be included in some embodiments of a test kit.

FIGS. 24A-24C illustrate example inserts according to some test kit embodiments.

FIGS. 25A and 25B illustrate example inserts of test kits with example components positioned therein in a testing configuration according to some embodiments.

FIGS. 26A and 26B illustrate example embodiments of a test kit and various features that can be included therein.

FIG. 27 illustrates an embodiment showing various components of a test kit.

FIGS. 28A and 28B illustrate embodiments of a sample tube and receptable for a test kit including various features that can be included thereon.

FIG. 29 illustrates embodiments of test kit components for a test kit.

FIG. 30 illustrates embodiments of test strips for a test kit including various features that can be included thereon.

FIG. 31 illustrates steps in an embodiment of a method for image preprocessing using a reference card.

FIG. 32 illustrates an example test kit having a stand and labeled areas according to some embodiments described herein.

FIG. 33 illustrates an example device stand that can be provided for medication monitoring according to some embodiments described herein.

FIG. 34 is a block diagram illustrating an example embodiment of a computer system configured to run software for implementing one or more embodiments of the health testing and diagnostics systems, methods, and devices disclosed herein.

FIG. 35 illustrates another example embodiment of a computer system configured to run software for implementing one or more embodiments of the health testing and diagnostics systems, methods, and devices disclosed herein.

### DETAILED DESCRIPTION

Although certain preferred embodiments and examples are disclosed below, inventive subject matter extends beyond the specifically disclosed embodiments to other alternative embodiments and/or uses and to modifications and equivalents thereof. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present technology.

Some embodiments herein are directed to health testing and diagnostics kits, as well as testing platforms related thereto. In some embodiments, the kits and/or platforms can facilitate remote health testing via remote connection of patients and medical providers. Remote or at-home medical testing provides various benefits over in-person visits to medical professionals. For example, remote or at-home medical testing provides both safety and convenience to patients and medical professionals. In-person visits by individuals with infectious diseases can endanger both medical professionals and anyone who encounters the individuals on their way to the in-person visit. Remote or at-home testing, in contrast, may not involve personal contact between the patient and any other individuals who may otherwise be at risk. Furthermore, at-home testing can be more convenient for many individuals, as neither medical providers nor patients need to leave the safety or comfort of their homes in order to administer or take a test using remote testing kits and platforms. In some cases, at-home testing may be done at any time of day on any day of the year. For example, at-home medical testing may be performed on weekends, holidays, at night, and/or at other times when an individual's regular doctor, urgent care provider, and so forth may be unavailable.

Additionally, because of advancements in medical and logistics technology, at-home testing can now be extremely fast. In some cases, diagnostic tests can be administered and read within seconds. Other tests may require a cure time before being read or may require delivery to a laboratory to receive results, but results can still be received within days in most cases.

Applications for remote or at-home medical testing are abundant. For example, remote or at-home testing can be used by travelers in any location to ensure that the traveler is healthy before and/or after arriving at a destination, without having to locate medical care in an unfamiliar locale. Furthermore, remote, or at-home testing may prevent the spread of infectious diseases by providing travelers knowledge about their health and their potential to transmit infectious diseases to others. This information may be used to inform a user's decisions to quarantine or avoid traveling altogether, and/or to avoid bringing home an infectious disease. Remote or at-home testing may also be useful for reducing stress and anxiety in sensitive individuals such as the elderly, chronically ill, and children. Remote or at-home testing may provide a better experience for such sensitive individuals, especially in cases in which the testing procedure is uncomfortable or invasive. Remote or at-home testing can mean that the test is done in a safe, comfortable, and familiar environment, so sensitive individuals may feel less stressed and worried during their test. This may allow testing to proceed more smoothly, may improve the overall experience of the user, and may lead to more frequent testing.

In some instances, remote or at-home testing can be performed in a user's home, although this need not be the case in all instances. For example, as used herein, remote, or at-home testing can refer to testing performed in other locations outside the home, such as in hotel rooms, airports, or other remote locations where access to an in-person healthcare provider is not available or desirable. Another consideration for remote or at-home testing is privacy. Remote or at-home testing can be private and discreet, which may be ideal for high-profile individuals or sensitive individuals who want to get tested without leaving their homes. Also, accessibility considerations favor remote or at-home testing. Remote or at-home testing can be ideal for anyone who has transportation issues or mobility/accessibility considerations.

Some embodiments herein are directed to a health testing and diagnostics platform for facilitating remote health testing via remote connection of patients and medical providers. Some embodiments are directed to test kit materials for facilitating remote medical testing. Some embodiments herein are directed to device stands that are configured to hold a user device, such as a smartphone, tablet, laptop, personal computer, or other type of personal device, during administration of a health or diagnostic test. The device stand can be configured to position the user device relative to one or more of the user, the test kit, or other testing materials during administration of the health or diagnostic test. In some embodiments, the device stand positions the user device such that one or more of the user, the test kit, or other testing materials are positioned with in the field of view(s) of one or more cameras of the user device (e.g., forward and/or rearward facing cameras). In some embodiments, one or more cameras may be built into the user device. In other embodiments, a camera can be external, for example connected using a USB connection. In some embodiments, the device stands are configured such that the user can still view a display on the user device so as to view testing information that is presented to the user on the display, such as a proctor, AR-based guidance, text, timers, or other testing instructions on the display of the user device while the user device is in the device stand. The device stands can thus facilitate remote or at-home medical testing.

In general, remote health testing and diagnostics can involve a user or patient interacting with a healthcare provider, automated system, etc., using a personal user device, such as a personal computer, a cellular phone, a smartphone, a laptop, a tablet computer, an e-reader device, an audio player, or another device capable of connecting to and communicating over a network, whether wired or wireless. In some embodiments, the healthcare provider can be a live person (e.g., a doctor, nurse, or other healthcare professional). In some embodiments, the user can interact with an automated system, such as pre-recorded step-by-step instructions or an artificial intelligence (AI) system. In some embodiments, remote healthcare can be provided by a mix of a live person, pre-recorded steps, and/or artificial intelligence systems.

In some embodiments, at-home healthcare testing or diagnostics can involve the use of a test kit. The test kit can include one or more items for administering a health or diagnostic test. In some embodiments, the user may film themselves taking the health or diagnostic test using the test kit using their user device (e.g., via a live online video stream and/or a recording stored locally on the user's device, which may be uploaded to a remote system and/or processed locally). In some embodiments, the user may also film the test kit during administration of the test. In some embodiments, the user may communicate with a healthcare professional using the user device during administration of the test. In some embodiments, the healthcare professional may be presented with video of the user using the test kit during administration of the test.

In some embodiments, the user can be instructed to position the test kit such that portions of the test kit that will be used during the testing process are within the view of the user device's camera and can be viewed by the user on the user device's screen. In this manner, guidance for how to perform the test can be presented overlaid onto the user's screen. The guidance can be provided using one or more of step-by-step instructions, text, demonstration videos, augmented reality (AR) content, schematic representations, and/or audible instructions. In one example, the user may need to add drops of a liquid to a specific portion of the test kit during administration of the test. Guidance in the form of an augmented reality animation can be overlaid onto the user's screen in a location corresponding to the displayed contents of the user's actual test kit to indicate where the user should place the drops of solution. As another example, when the user needs to access a swab within the test kit, the location of the swab can be highlighted using AR on the user's screen.

In some embodiments, it may be desirable to position the user device relative to the user and the test kit in a specific orientation, for example, in a specific orientation that allows for the user and the test kit to be within view of at least one camera on the user's device and that allows for the user to easily see the display screen of the user device. It may further be desirable to position the user device relative to the test kit within the field of view of the user device's camera such that guidance (e.g., AR guidance) can be overlaid thereon.

In some embodiments, both a user's face and test kit materials must be within the FOV of a camera of a user device. In some embodiments, a user's face must be within the FOV of a camera of a user device. In some embodiments, test kit materials must be within the FOV of a camera of a user device. In some embodiments, specific items from a test kit must be within the FOV. In some cases, users of remote medical testing may encounter difficulty ensuring that the user, the testing kit materials, or both are within the field of view of a camera. Augmented reality and/or computer vision techniques may be used to assist users with remote medical testing.

In some embodiments, augmented reality and/or computer vision techniques may be used to guide a user to position the user's face and/or another part of the user's body within the FOV of a camera. In some embodiments, augmented reality and/or computer vision techniques may be used to guide the user to place test kit materials within the FOV of a camera. In some embodiments, the user may be guided to place the user's face in a particular region of the FOV. In some embodiments, the user may be guided to place the test kit materials in a particular region of the field of view. For example, in some embodiments, a user may be directed to place the user's face inside a first bounding box. In some embodiments, a user maybe directed to place test kit materials inside a second bounding box. While the term “bounding box” is used herein, it is not intended to be limiting. It will be understood that a bounding box could be any shape such as, for example, a square, a rectangle, a circle, an oval, a triangle, or any other two-dimensional shape. In some embodiments, a bounding box may represent the shape of a body part or one or more test kit materials.

In some embodiments, a stream of image frames may be captured by a camera of a user device. In some embodiments, one or more images of the stream of image frames may be modified with augmented reality content which may include, for example, one or more bounding boxes. In some embodiments, a surface in the FOV may be identified, for example a tabletop. In some embodiments, a bounding box may be overlaid onto an identified surface. In some embodiments, a stream of image frames may be displayed on a display of a user device. In some embodiments, a stream of modified image frames containing one or more bounding boxes may be displayed on a display of a user device. The bounding box may be used to, for example, indicate where a user should place testing materials.

In some embodiments, a machine learning model may be trained to recognize test kit materials. In some embodiments, a machine learning model may recognize a specific test based on, for example, the test kit packaging. In some embodiments, a machine learning model may recognize one or more test kit materials. In some embodiments, a machine learning model may be trained to recognize a changed test kit such as, for example, if a manufacturer updates its packaging. In some embodiments, a machine learning model may be trained to recognize a planar area such as, for example, a table surface. In some embodiments, a machine learning model may be trained to recognize a face or other body part.

In some embodiments, the systems, methods, and devices described herein may be configured to determine whether a face of a user associated with the user device is positioned within a first bounding box. In some embodiments, the systems, methods, and devices described herein may be configured to determine if one or more medical diagnostic testing materials are positioned within a second bounding box. In some embodiments, more than two bounding boxes may be used. In some embodiments, the user may be instructed to move the face of the user, another body part of the user, and/or one or more medical diagnostic testing materials. In some embodiments, in response to determining that the face of the user associated with the user device is positioned within a first bounding box and determining that one or more medical diagnostic materials are positioned with a second bounding box, the systems, methods, and devices described herein may perform one or more operations to guide the user through a testing procedure. In some embodiments, the user may be shown an indication that a face and/or other body part is within a bounding box. In some embodiments, an indication may be, for example, an audio notification, a display on the screen such as, for example, a checkbox, an animation, or some other indication.

In some embodiments, the user may move out of a bounding box and/or out of the FOV of the camera of the user device. In some embodiments, the user may be prompted to move back within the FOV of the camera and/or back within the bounding box. In some embodiments, the user may move one or more diagnostic testing materials out of a bounding box and/or out of the FOV of the camera of the user device. In some embodiments, the user may be prompted to move the one or more diagnostic materials back into the FOV of the camera and/or back within the bounding box. In some embodiments, a prompt may consist of text, audio, and/or video, or some other means of notifying the user that action is needed.

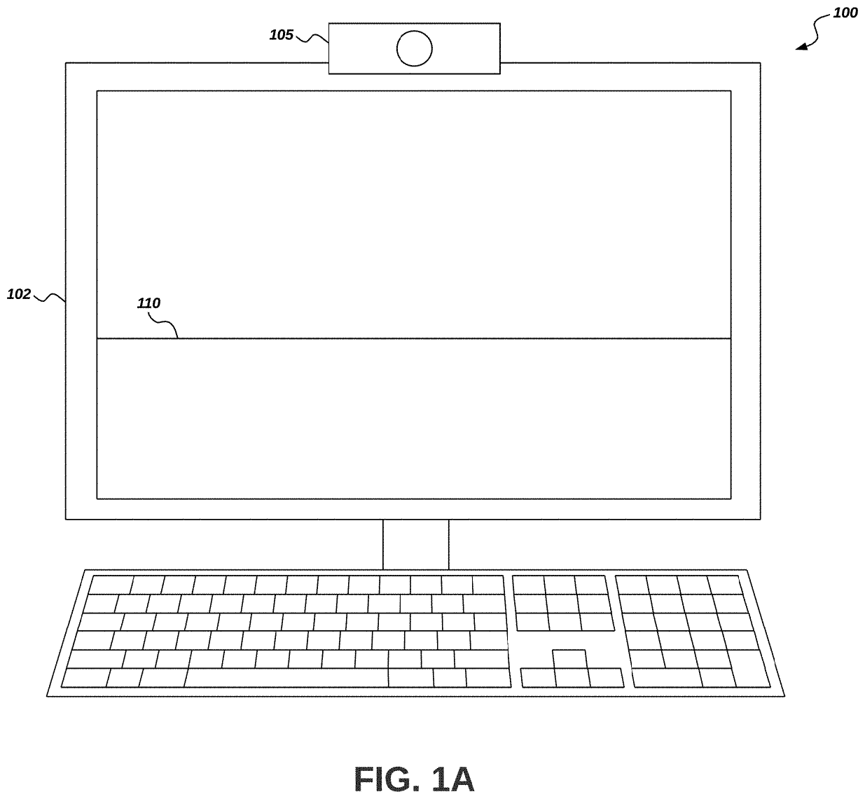

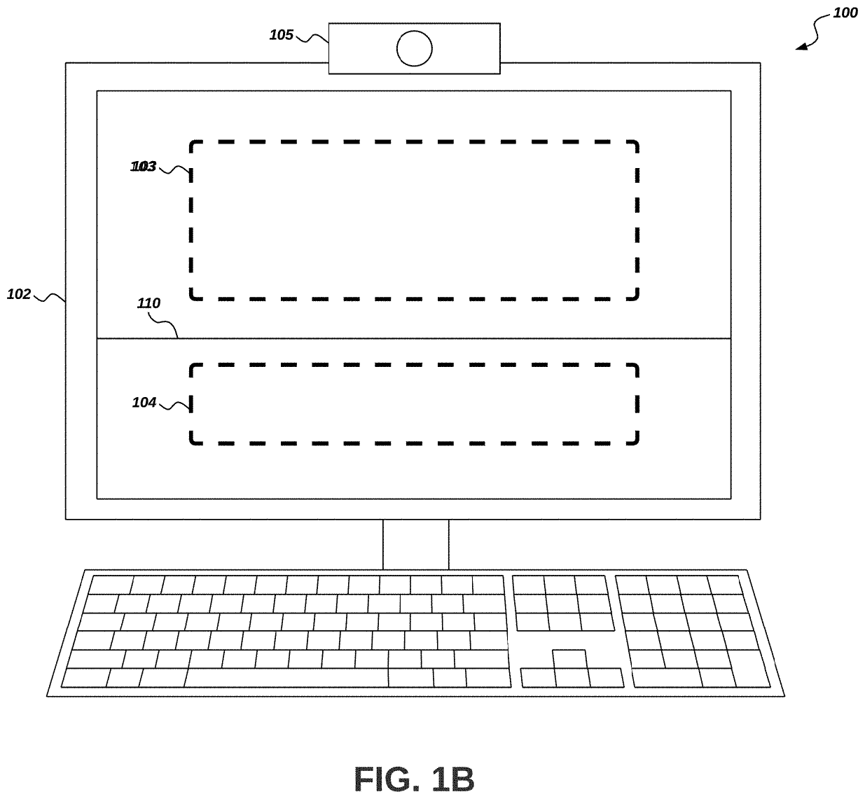

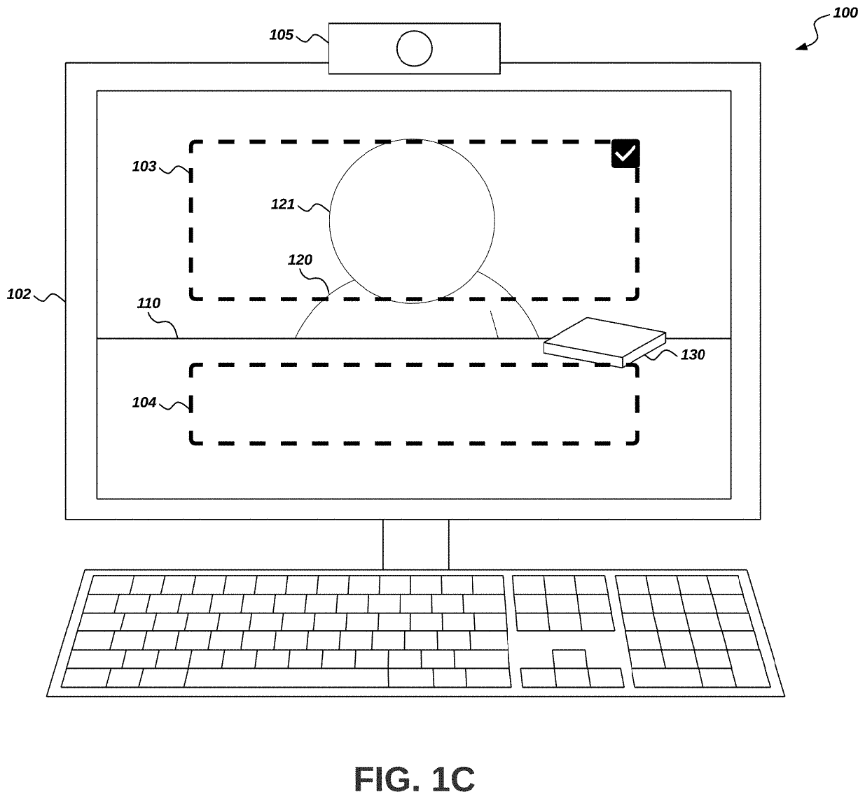

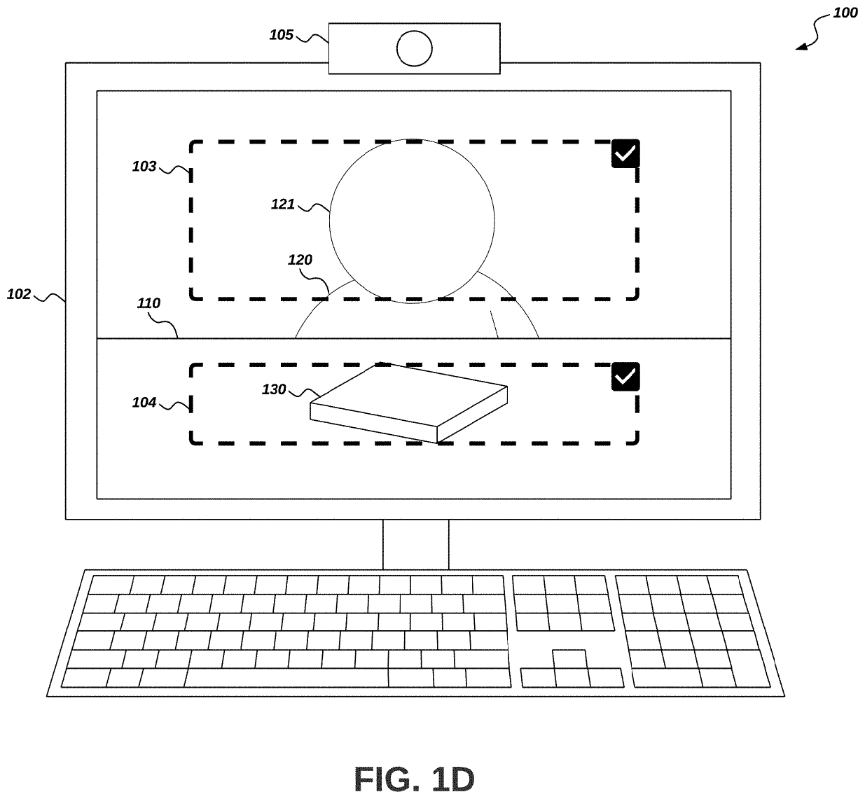

With reference to FIG. 1A, in some embodiments, a user device 100 may have a camera 105 and a display 102. The display may show a video stream on the display 102 that is being captured by the camera 105. In some embodiments, the video stream may show a surface 110 which may be, for example, a table on which the user device 100 is resting. In some embodiments, the surface 110 may be identified, e.g., using a machine learning model trained to recognize planar areas. According to FIG. 1B, in some embodiments, the video captured by the camera 105 and shown on the display 102 may be modified to include augmented reality content including a first bounding box 103 and a second bounding box 104. As depicted in FIG. 1B, in some embodiments, the second bounding box 104 may be overlaid onto the identified surface 110. More specifically, in these embodiments, one or more characteristics of the second bounding box 104 (e.g., position, size, geometry, etc.) may be determined based on one or more characteristics of the identified surface 110 (e.g., position, size, geometry, etc.). That is, the video captured by the camera 105 and shown on the display 102 may be modified based at least in part on one or more characteristics of the identified surface 110. According to FIG. 1C, in some embodiments, a user 120 may place the user's face 121 inside the first bounding box 103. In some embodiments, a diagnostic test kit 130 may be outside a second bounding box 104. According to FIG. 1D, in some embodiments, a diagnostic test kit 130 may be moved within a second bounding box 104. In some embodiments, the user may be shown confirmation that the face 121 and the diagnostic test kit 130 are placed within the appropriate bounding boxes 103 and 104. In some embodiments, in response to a determination that the face 121 is positioned within the first bounding box 103 while the diagnostic test kit 130 is simultaneously positioned within the second bounding box 104, an execution of one or more operations may be initiated to guide the user 120 through a testing procedure. For example, if the testing procedure involves the user collecting a biological sample from their nose, then such one or more operations may include instructing the user 120 to remove a swab from the diagnostic test kit 130 and insert the swab into one or both nostrils of the user 120. In at least some of these embodiments, the determination of whether the face 121 and the diagnostic test kit 130 are placed within the appropriate bounding boxes 103 and 104 may further include a determination of whether the depths at which the face 121 and the diagnostic test kit 130 are positioned satisfy one or more threshold values (e.g., depth values that are associated with bounding boxes 103 and 104). In some embodiments, display of one or both of the first and second bounding boxes 103, 104 may be discontinued upon execution of such one or more steps. In some embodiments, the video captured by the camera 105 and shown on the display 102 may be modified to include augmented reality content including more than two bounding boxes. For instance, a third bounding box may be presented and correspond to a region within which the user 120 is to place or position a driver's license, passport, or other identification credentials. Although described primarily within the context of remotely administered medical testing, it is to be understood that one or more of the systems, methods, and devices described herein may be leveraged in any of a variety of other applications in which remote proctoring and/or verification services may be desirable and/or necessary (e.g., online education, user identity verification for online dating and/or e-commerce, etc.). The systems, methods, and devices described herein may also be used in other contexts, such as aiding users in complying with medication regimens and/or verifying that users are in compliance with medication regimens.

While the examples in FIGS. 1A-1D depict a single display 102 and camera 105, such as may be encountered when a user is using a desktop computer, laptop, etc., other configurations are possible. For example, the user device can be a tablet, smartphone, convertible PC, and so forth having multiple cameras. For example, a user device can have a frontward-facing camera and rearward-facing camera.

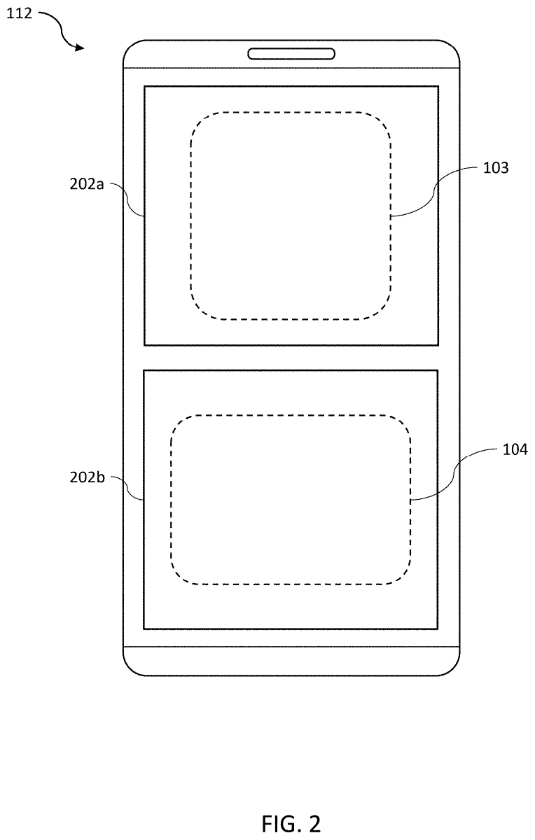

FIG. 2 shows an example embodiment of an interface in which two camera views are shown. In FIG. 2, the user device 112 can be, for example, a smartphone having a frontward-facing camera and a rearward facing camera. The image captured by the frontward-facing camera can be displayed in a first view 202a, which can include a first bounding box 103. An image capture by the rearward-facing camera can be displayed in the second view 202b, which can include a second bounding box 104. In some embodiments, the first view 202a and second view 202b can be the same size or can be different sizes. In some embodiments, the sizes of the first view 202a and second view 202b can change during the a testing process, for example to provide the user with a better view of test components (for example, during a step in which the user is working with the test components, such as adding drops to a test strip) or a better view of the user (for example, when the user is collecting a sample).

As discussed above, it can be important to ensure that the user, test kit materials, or both remain in view of one or more cameras during a testing session. Users may encounter difficulty placing test kit materials and/or themselves in appropriate locations and maintaining proper positioning through the testing session. Accordingly, in some embodiments, a test kit can be designed in a way that aids the user in properly setting up their environment.

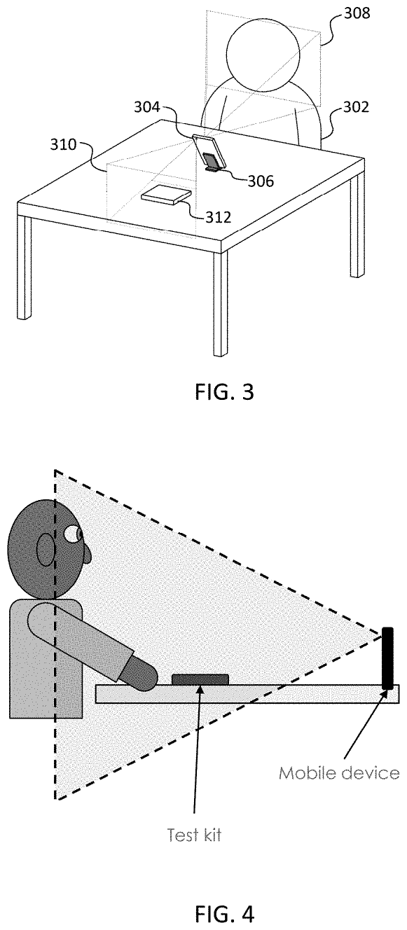

FIG. 3 illustrates an example setup of a user 302, a user device 304 (e.g., a smartphone) and a test kit 312 during administration of a health or diagnostic test performed using the user device 304 and the test kit 312. In the illustrated example, the user 302 accesses the health testing and diagnostic platform using the user device 304. In this example, the user device 304 includes both forward-facing and rearward-facing cameras. One or both of these cameras can be used during a testing procedure to capture images of, for example, the user 302 and/or the test kit 312 used during the testing procedure. Further, the images captured by the forward and/or rearward facing cameras can be displayed to the user on a display of the user device 304. The display of the smartphone may be located on the front surface of the user device 304 along with the forward-facing camera. Moreover, AR-based guidance can be added to the images displayed to the user to facilitate and improve the testing experience.

The relative positioning between the user 302, the user device 304, and the test kit 312 as illustrated in FIG. 3 may be advantageous in some embodiments. In the illustrated example, the user 302 sits at a table, desk, countertop, floor, or other large, flat surface. The test kit 312 is also positioned on the table in front of the user. The user device 304 is positioned on the table between the user 302 and the test kit 312. In the illustrated embodiment, the user device 304 is supported by a stand 306, which can, for example, be a component included in the test kit 312. The user device 304 is positioned (in some embodiments, with the assistance of the stand 306) such that the user is visible within the field of view (FOV) 308 of the user device's forward-facing camera and the test kit 312 is positioned within the FOV 310 of the user device's rearward-facing camera. Such a setup may be advantageous as it allows the user 302 and the test kit 312 to remain within the different FOVs of the forward- and rearward-facing cameras of the user device 304 during the entire testing procedure. Further, at different portions of the procedure, the output of the forward- and/or rearward-facing cameras can be displayed to the user via the user device screen. In some embodiments, said output may be supplemented with on-screen AR-based guidance for completing the testing procedure.

For example, during a first portion of the testing procedure, the output of the rearward-facing camera (e.g., FOV 310 in which is positioned the test kit 312) can be displayed to the user such that the user can view the test kit 312 on the display of the user device 304. The display can be updated with AR-based guidance to highlight certain areas of the test kit 312 or items in the test kit 312. The real-time video display of FOV 310 as presented on the user device's screen may additionally or alternatively be overlaid with other types of instructions to aid the user in performing the testing procedure. Audio instructions may be included and may be presented to the user by speakers on the user device 304. During another portion of the testing procedure, the output of the forward-facing camera (e.g., FOV 308 in which the user 302 is positioned) can be displayed to the user such that the user can view himself or herself in real time on the display of the user device 304. The real-time user video displayed to the user can be modified to include AR-based guidance to highlight certain areas of the user (e.g., a nostril) and/or may be overlaid with other types of instructions to aid the user in performing the testing procedure.

In some embodiments, the setup illustrated in FIG. 3 and/or use of the stand 306 can facilitate setting up standard distances between the user 302, the user device 304, and the test kit 312. In some embodiments, the system may benefit from detecting, measuring, or otherwise knowing the distances between user 302, the user device 304, and the test kit 312. For example, knowing these distances may allow the system to more easily identify certain test kit components or other elements within the field(s) of view 308, 310 of the cameras and/or may ensure that all steps of the testing procedure that must be observed are accurately captured by one or more cameras. In some embodiments, the stand 306 can be integrated into the test kit box. For example, a portion of the test kit box can fold out to provide the stand and fix the distance between the test kit and the user device as will be discussed in further detail herein below.

Although the setup illustrated in FIG. 3 makes use of forward- and rearward-facing cameras on the user device 304, in other examples, only cameras on one side of the smartphone (e.g., front or back) may be used. For example, as shown in FIG. 4, the user may place the smartphone in a stand, and may be instructed to position the test kit in front of the user device (e.g., smartphone) such that both the user and the test kit are visible in the forward-facing camera on the front of the smartphone above the screen. In such an example, one or both of the user and the test kit can be positioned within the forward-facing field of view of the smartphone as shown in FIG. 4.

Use of a device stand, for example, as illustrated in FIGS. 3 and 4, can be advantageous in that, in some testing instances, the user needs to be “hands free” to manipulate the test contents. For example, it may be difficult for a user to hold the user device while also performing administration of the test. Use of the device stand can free up the user's hands. Additionally, use of the device stand can allow the user and the testing kit to remain constantly within the field of view(s) of the user device during administration of the test, which may facilitate test continuity. For example, in some embodiments, the user and/or the test kit remain in view of the cameras of the mobile device during administration of the test such that a proctor can monitor the entirety of the tests (or key portions of the test which require monitoring).

In some embodiments, it may be advantageous to use a single user device position that can persist throughout the test. For example, this can allow the user and the test kit to be seen simultaneously, ensuring that the same user persists through the test while all steps are completed. Maintaining the user and the test kit within view of the user device throughout the testing session may improve test security and may also bolster validity of the test results. Additionally, in some embodiments, maintaining the user device in a single position improves the user experience by reducing testing complexity. For example, the user will not need to reposition the phone one or more times during the test if the user device remains positioned in a stationary position using a stand.

In some embodiments, the device stands are configured such that they do not occlude or block the rear camera of the user device. This can allow the rearward-facing camera of the user device to be used to scan a QR code, assist in reading test results, etc., without having to reposition the device.

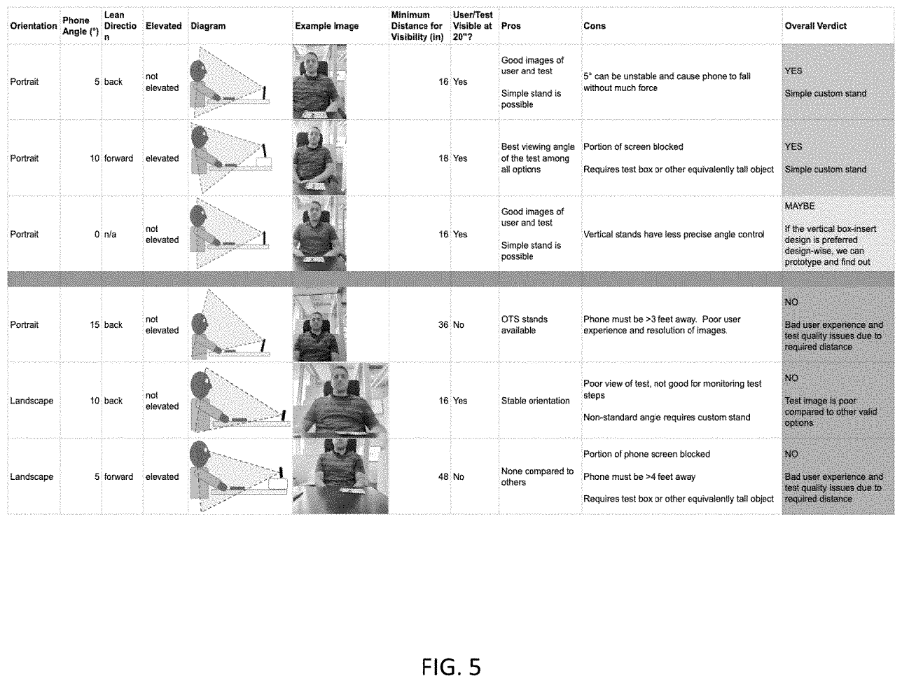

FIG. 5 illustrates results of a study that was completed of multiple different options for device stands to be used during remote health or diagnostic testing. As shown, various parameters were tested, including orientation, user device (e.g., smartphone) angle, lean direction, and user device elevation (e.g., relative to the surface on which the test kit is placed). As shown, the study determined that, in some embodiments, (1) a portrait orientation with a 5° lean back, and without elevating the phone, or (2) a portrait orientation with a 10° lean forward, and with the phone elevated, produced the most positive results, providing good views of the user and test kit when the user and the test kit are on the same side (e.g., a front side) of the user device. It was also determined that a portrait orientation with the phone oriented vertically (i.e., with 0° lean), and with the phone not elevated, may also be suitable. FIG. 5 illustrates these three user device positions, including example schematic versions of forward-facing field of views and sample images, in more detail. The results of these tests should not be considered limiting, as other orientations, leans, elevations, etc., may also prove advantageous in some conditions and with various types of user devices. For example, a relatively tall user device (e.g., a tablet or a smartphone with a relatively large screen) may perform best at different orientations, leans, elevations, etc., than a relatively small device.

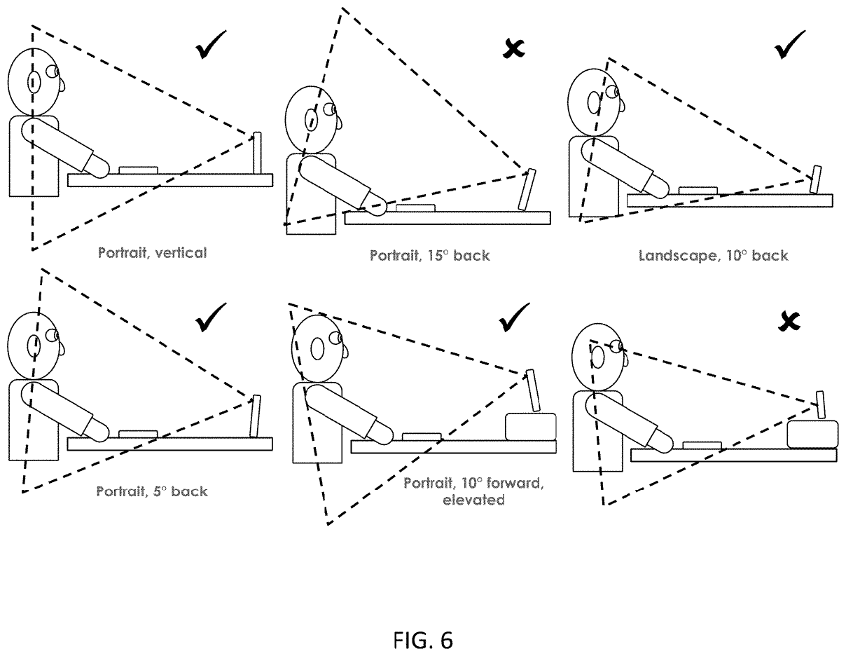

FIG. 6 illustrates the field of views of a camera of a user device positioned with different orientations, angles, and elevations. In these examples, field of views that are desirable (e.g., that are able to capture both the user and the test kit using a forward-facing user device camera) are indicated with check marks, while forward-facing user device camera field of views that fail to sufficiently capture at least one of the user or the test kit are indicated with X marks. In some embodiments, forward-facing smartphone cameras generally have a field of view of about 54° vertically and about 41° horizontally when positioned in a portrait orientation. Accordingly, it may generally, though not necessarily, be advantageous for a smartphone to be oriented in a portrait orientation, especially when the forward-facing camera is used to capture both the user and the test kit, as the user and test kit will generally be at least roughly horizontally aligned, while there may be a significant vertical distance between the test kit (which may be, for example, on a table or desk) and the user's head.

In some embodiments, factors that may be relevant to the design of a mobile device stand for use with remote or at-home health or diagnostic testing can include the complexity of the stand design, portability of the stand, reusability of the test, the desired distance between the user device and the user, the desired distance between the user device and the test kit, the number and positions of the cameras on the user device that are used during a testing procedure, the size and weight of the user device, as well as the type of testing to be performed, among others. In some embodiments, a test kit can be designed to enable consistent placement of test components, the user device, and so forth, which may enable a smoother user experience and reduce the likelihood of an error being made during a testing procedure.

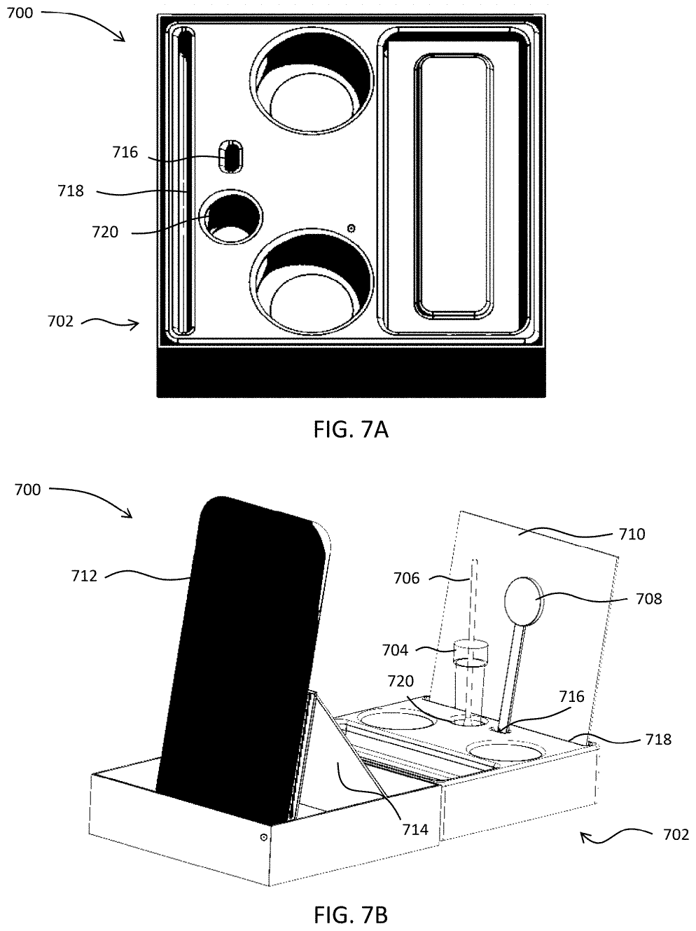

FIGS. 7A and 7B illustrates an embodiment of a portion of a test kit 700 and various features and components that can be included therein. As shown in FIGS. 7A and 7B, the test kit 700 can include, for example, packing 702, a sample tube 704, a swab 706, a test strip 708, and a reference card 710. The packaging 702 may include an inner box (e.g., a bottom half of a box) and an insert disposed within the inner box. The packaging 702 can be configured to hold the sample tube, the swab, the test strip, and the reference card in a configuration that facilitates testing. In the illustrated example, the packing also positions a user device 712, such as a smartphone, relative to the other materials to facilitate testing. The user device 712 may be positioned using a user device stand 714. The user device stand 714 may be integrated with or otherwise included with the packaging or may be a separate component not included with the test kit. The user device stand 714 may be configured to hold the user device 712 such that a camera on the user device is oriented toward the test strip 708 and the reference card 710 when the test strip 708 and reference card 710 are placed within a test strip slot 716 and a reference card slot 718, respectively. When the user device 712 is supported by the user device stand 714, the camera on the user device is configured to capture an image that includes at least the test strip 708 and the reference card 710. Additional features within the packaging may also be included. For example, a sample tube slot 720 may be included to hold the sample tube 704 and the swab 706 as shown.

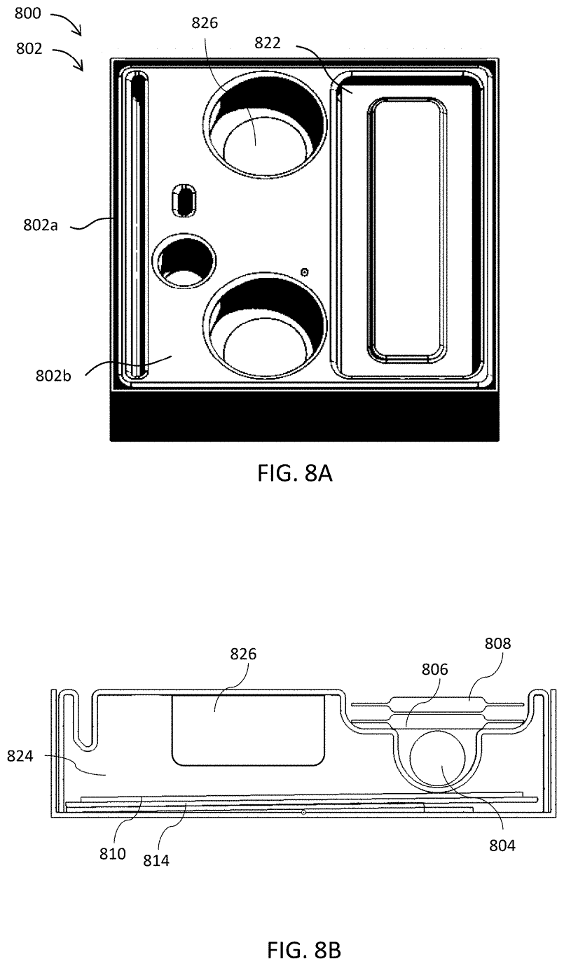

FIGS. 8A and 8B illustrate an embodiment of a portion of packaging (e.g., an inner box and an insert disposed therein) that can be included in a test kit as well as various features that can be included therein. In the illustrated example of FIGS. 8A and 8B, the insert is configured with slots for the reference card, as well as dual slots for the sample tube and test strip. The various slots may be similar to or the same as those illustrated and described with respect to FIGS. 7A and 7B. When in the testing configuration, for example, as shown in FIG. 7B with various test kit components placed in their respective insert slots, the sample tube and test strip can be positioned in front of the reference card 810. Other arrangements are also possible, for example, as shown in several additional examples described throughout the application. FIG. 8A illustrates a top perspective view of a portion of a portion of test kit box 800 that includes packaging 802. The packaging 802 may include a portion of a box 802a (e.g., a lid or a base of a box) and an insert 802b configured to nest within the lid or base of the box. In some embodiments, the insert 802b may be fixed to the portion of the box 802a or may be integrally formed therewith. The insert 802b may be formed having features discussed above with respect to FIGS. 7A and 7B (e.g., a plurality of slots configured to receive a reference card, a sample tube, a test strip, a nasal swab) that are used during the testing procedure. In some embodiments, the insert 802b may include one or more recesses that are configured to receive and secure one or more of the test kit components during storage and/or shipping. For example, referring to FIGS. 8A and 8B, the recess 822 may be configured to hold a sample tube 804 when the sample tube is lying on its side.

The recess 822 may further receive a packaged swab 806 and/or a packaged test strip 808. The insert 802b may include a hollow bottom volume 824 between a top surface of the insert and the packaging portion of the box 802a. Within the volume 824, additional test kit components may be stored such as the reference card 810, folded user device stand 814, and/or instructions or other advertising or informational material (not shown). In addition to recesses configured to hold various test kit components at one or more stages of test storage, shipping, and use, the insert 802b may include one or more recesses 826 configured to assist a user in removing the insert 802b from the box 802a. The recesses 826 may be configured to receive a user's thumb and finger as the user makes a pinching motion and removes the insert from the box. This allows the user to lift the insert out of the box without dumping out the contents in a way where components could be lost or contaminated.

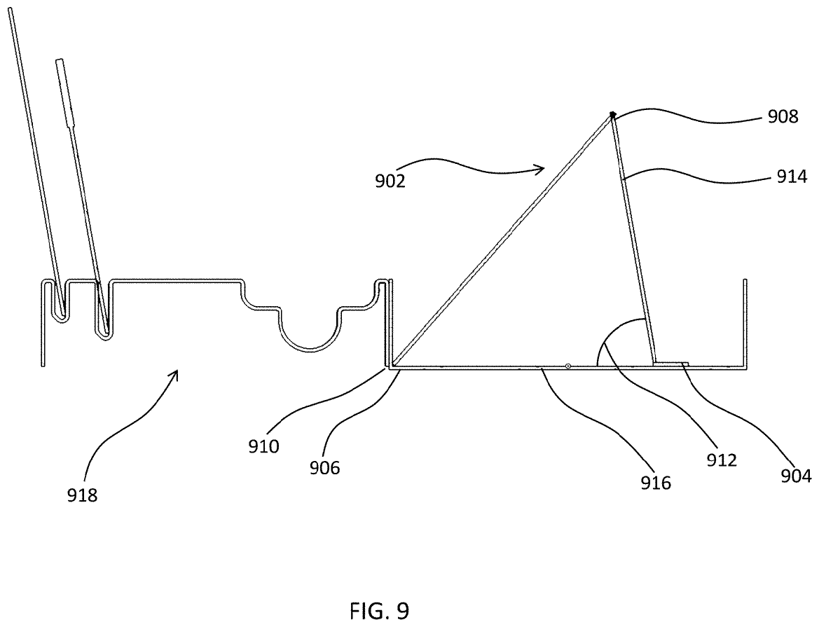

FIG. 9 illustrates embodiments of a test kit in an example testing configuration. In this example, a portion of the box (e.g., the lid or the base of the box) can include a piece that folds to form a stand for holding the user device (e.g., a smartphone). In the embodiment shown, the piece 902 that folds to form a stand includes a first end 904 and a second end 906, wherein the fold 908 is located between the first and second ends. One of the ends (e.g., the first end) may be fixed to the box using adhesive, hook, and loop, or other mechanical or chemical, permanent, or selectively removable means. The other side may be propped or wedged against a peripheral side or corner 910 of the box so that the second end does not slide relative to the first end when the user device is placed on the stand. The angle 912 of a first leg 914 of the stand relative to the base of the box 916 in which it is assembled may be selected such that a user device resting against the first leg 914 is positioned to capture an image (e.g., using a rearward-facing user device camera) that includes all test kit components placed in the insert 918.

Below, various other designs and configurations for mobile device stands will be discussed with reference to the examples illustrated in the figures. It will be appreciated that other stand designs are also possible based upon the examples and principles described herein. Accordingly, this disclosure should not be limited to only the illustrated examples.

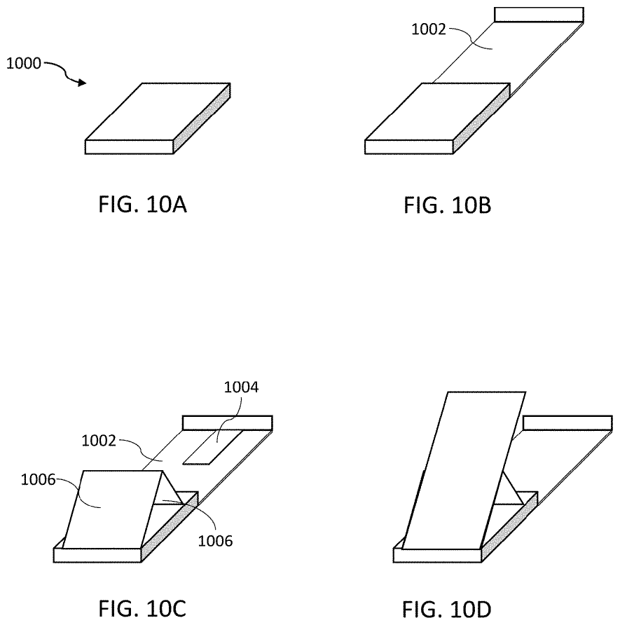

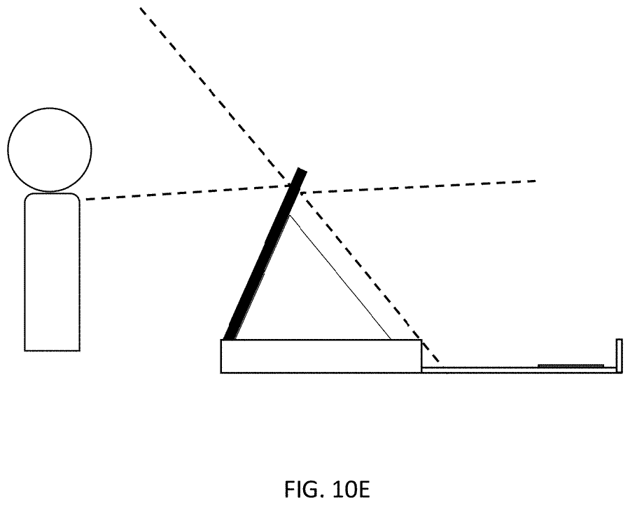

FIGS. 10A-10E provide details of an example device stand that can be integrated into the packaging of a health or diagnostic test kit. For example, FIG. 10A illustrates the kit 1000 in a closed configuration (e.g., a closed package or box). FIG. 10B illustrates that a lid of the kit can be configured to open. In some embodiments, the lid flips open such that it remains attached to a bottom portion of the box. In the illustrated embodiment, the kit includes the materials necessary for performing the test, including reagents, swabs, etc. FIG. 10C illustrates that the open lid 1002 may provide a test area 1004 or surface on which one or more portions of the test can be performed (for example, the lid can include a lateral flow test strip). In some embodiments, the open lid 1002 can include markings to indicate where test kit components (such as a test strip) can be placed. As shown in FIG. 10C, the contents of the test kit can be removed, and the test kit packaging can be folded to produce a device stand. In some embodiments, the two legs 1006 of the stand illustrated in FIG. 10C may initially be placed in a folded position such that the two legs 1006 are stacked flat on top of each other (i.e., an inside angle between the two legs is substantially zero) and the stand lays flat at the bottom of the box in order to save space inside the box. To erect the stand, the two legs may be unfolded by the user to form a wedge or triangular shape with the first leg supported against one side of the bottom portion of the box and the second leg supported against a second side of the bottom portion of the box. The first and second sides can be opposite each other. In the illustration of FIG. 10D, one side of the bottom portion of the box against which the stand rests can be adjacent to the area of the box where the bottom portion is attached to the flipped open lid. This configuration can allow for the rearward-facing camera of a user device positioned on the stand to be aimed at the flipped open lid. For example, FIG. 10D illustrates a smart phone or other user device positioned on the stand. FIG. 10E illustrates that the user can be positioned within a forward-facing field of view of the user device and the test area provided on the open lid of the kit can be positioned within a rearward-facing field of view of the user device, in a manner similar to that shown in FIG. 3. In some embodiments, the size of the stand legs and the angle between the stand legs when the stand is in the assembled position may be selected such that the stand leg configured to receive the user device (e.g., the stand leg supported by the portion of the box that is not attached to the flipped open lid) places the user device with an orientation between vertical and 20 degrees of backward lean, for example 0 degrees, 5 degrees, 10 degrees, 15 degrees, or 20 degrees, or any number between these numbers, or even more if desired. In some embodiments, the box can include grooves or indentations that can be used to set the angle of the stand leg configured to receive the user device, for example to accommodate devices with cameras in different locations. The stand legs may have the same or different size dimensions. Additionally, while both legs are described as being supported by sides of the box, other embodiments are possible wherein only one of the two stand legs is supported by the box while the other of the two stand legs does not require lateral support from the box.

In some embodiments, the mobile device stand can be included in the kit (e.g., as a discrete component), or it can be integrated into the box or other packaging that makes up the kit materials (e.g., as a foldable structure). For example, in the embodiment described with respect to FIGS. 10A-10E, one of the two legs of the stand may be tethered, attached, or otherwise movably fixed to the lower portion of the box so that the stand can be easily unfolded for use and folded for storage without risk of the stand falling out or being misplaced.

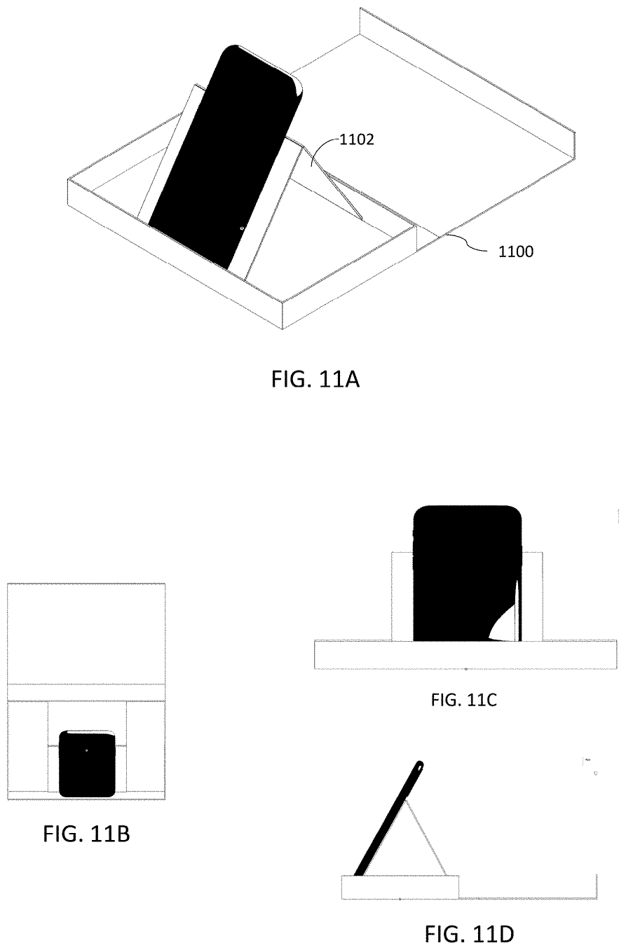

FIGS. 11A-11D illustrate another example of a user device stand that can be easily and cheaply manufactured into the packaging of a health or diagnostic test kit. Such a stand and/or kit can be useful for a diagnostics test, prescription medication, or various other at-home healthcare items. In the illustrated example, the device stand comprises a rigid or semi-rigid sheet that can be folded into a wedge 1102 which can be supported by the sides of the packaging 1100 (e.g., a lower portion of the box). This configuration can be advantageous as, by switching between the forward- and rearward-facing cameras, all views needed for proctoring and test completion can be available from a single, stationary phone position. Eliminating the need to move or reposition the user device allows the user's hands to remain free to handle test kit components. Additionally, test information will appear in relatively the same location in the image regardless of type of user device (e.g., regardless of the make or model of smartphone), thereby making proctor guidance and computer vision more predictable and simpler. In the illustrated configuration, the forward-facing camera captures the user's face and certain interactions between the user and test components (e.g., a nasal swabbing process), and the rear camera observes the test components, various test steps (e.g., the user dropping reagent from a reagent bottle onto a test strip and/or the user transferring the collected sample onto the test strip), test results, printed codes, etc. In some embodiments, at one or more instances during the test, a display on the front of the user device may show the output of the forward- or rearward-facing cameras. In some embodiments, bounding boxes, test instructions or other guidance (e.g., AR-based guidance) can be overlaid thereon.

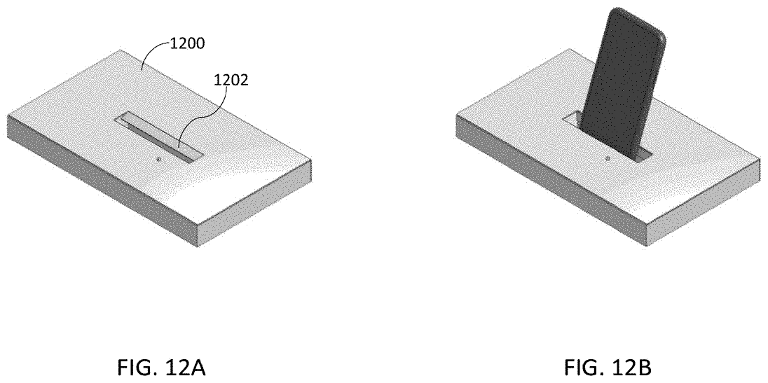

FIGS. 12A and 12B illustrate an additional mobile device stand concept. In the illustrated embodiment, the stand 1200 comprises a slot 1202 configured to receive the user device. The slot 1202 can be configured on a lid or other surface of a test kit. In some embodiments, the slot 1202 may be included in a small box that is separate from and stored within the test kit packaging. When the user device is placed within the slot 1202, the device may be positioned in a forward- or backward-leaning orientation depending on the way the user sets up the device. This adjustability may provide broad functionality for various types and brands of user devices that have cameras in different areas of the device or that have cameras with different fields of view. Additionally, the slot 1202 may be configured such that the bottom of the user device is supported by a step or other feature of the stand that causes the user device to be elevated relative to a surface on which the box or packaging is placed. As discussed above, elevating the user device may improve visibility of the user and/or the test kit to the user device.

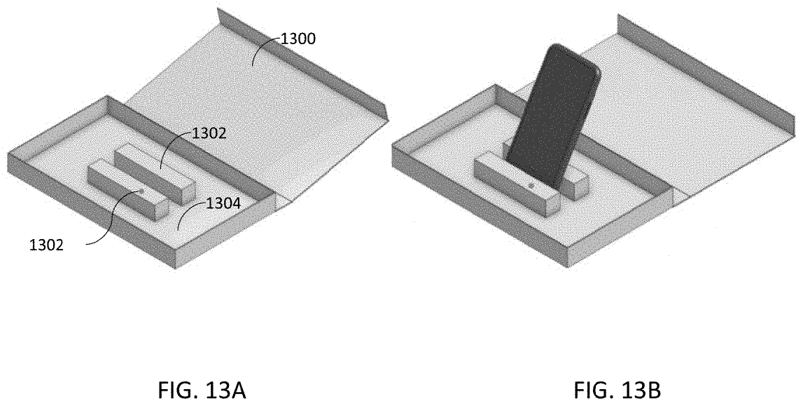

FIGS. 13A and 13B illustrate an additional mobile device stand concept. In this example, the stand comprises two blocks 1302 (e.g., foam or cardboard blocks) secured to a lower surface 1304 (or another surface) of the test kit packaging 1300. The phone can be positioned between and held by the blocks 1302 as shown. Similar to the configuration of FIGS. 12A, 12B, the user device may be adjustable between a variety of angles depending on the relative position between the two blocks 1302. Additionally, while not shown, an additional step disposed between the two blocks 1302 may be included to elevate the user device. In some embodiments, the blocks 1302 may be fixed in place, for example by gluing. In some embodiments, the one or more of the blocks 1302 can be movable. For example, a user may be able to adjust one or more of the blocks 1302 and fix them in place, for example using double-sided tape, hook, and loop fasteners, and so forth.

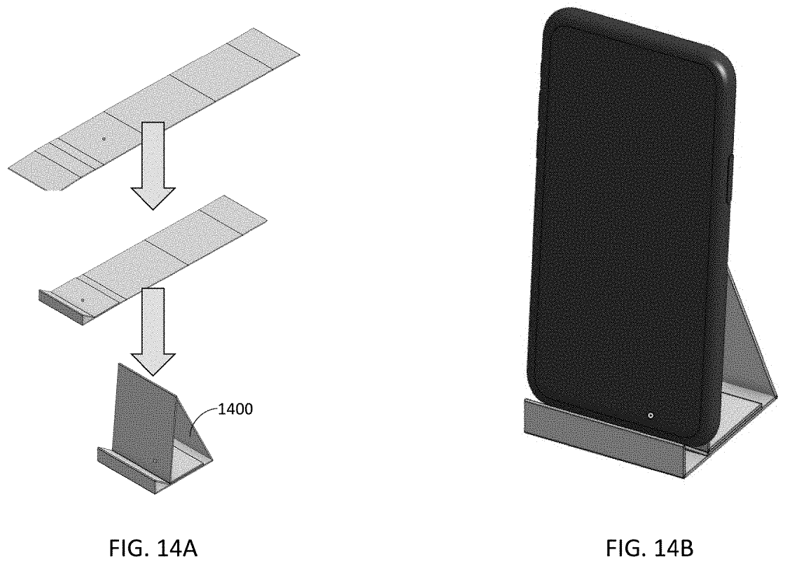

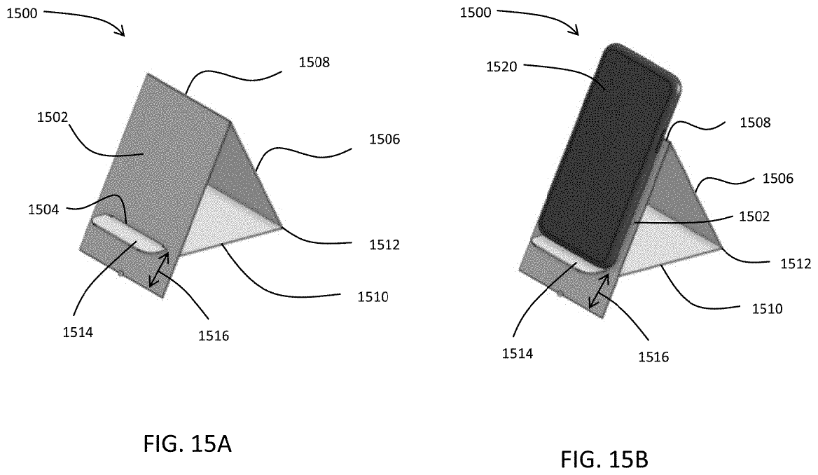

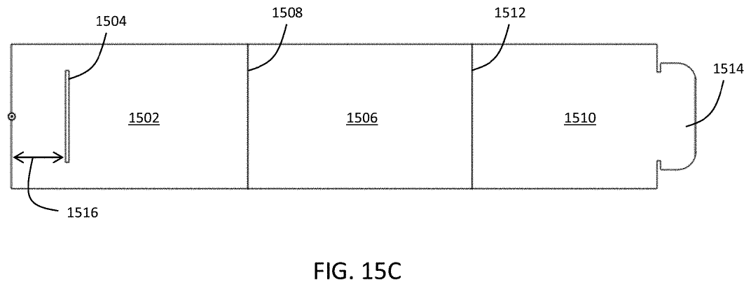

FIGS. 14A-14B and 15A-15C illustrate user device stands that can be configured to be foldable. These stands can be provided with an initially flat configuration (which may be beneficial for packaging and shipping) and can then be folded into a three-dimensional shape configured to hold a user device at a desired orientation and angle. In some embodiments, these stands can be formed from a sleeve, such as marketing material wrapped around a test kit box. The stand may also be configured such that the back supporting portion does not obscure the view of the rearward-facing camera on the user device. This may be accomplished by, for example, providing a cutout or by ensuring that the height of the back supporting portion of the stand is shorter than the distance between the base of the user device and the rearward-facing camera. The stand embodiment illustrated in FIGS. 14A-14B may maintain its structure when the user device is placed therein. For example, the weight of the user device may prevent the stand 1400 from unfolding.

The stand configuration 1500 illustrated in FIGS. 15A-15C includes a slot through which a tab on one end of the stand 1500 is inserted and held in place. The stand 1500 may include a first section 1502 having a slot 1504 therein. The slot 1504 may be located a distance 1516 from the end of the stand 1500 and the distance 1516 may be selected as a matter of design choice in order to achieve a desired elevation of a user device 1520 when the stand 1500 is assembled. The first section 1502 may be separated from a second section 1506 by a crease 1508. The crease 1508 may be a perforated line, a printed line, an indentation, and/or other mark or feature indicating to the user where the sheet should be folded. Similarly, a third section 1510 of the stand can be separated from the second section 1506 by a second crease 1512. The third section 1510 may include a tab 1514 configured to protrude through the slot 1504 and may form a shelf upon which the base of the user device 1520 rests. Such an embodiment may allow for the user device to be elevated relative to a surface on which the testing kit is placed. In some embodiments, multiple slots may be provided on the first section of the foldable stand such that the user can select an amount of elevation based on which slot the user inserts the end of the stand through. Such an embodiment may provide adjustability for different user devices such that each user is able to achieve an optimal rearward-facing and/or forward-facing angle and orientation. In some embodiments, the foldable test stand can be made from paper, cardboard, cardstock, plastic, or other similarly semi-rigid or foldable materials. In some embodiments, such foldable stands can be provided in a health or diagnostic test kit, may be formed from a portion of the test kit box, or may be formed from a sleeve, flyer, or other marketing material covering or otherwise coupled with the test kit box.

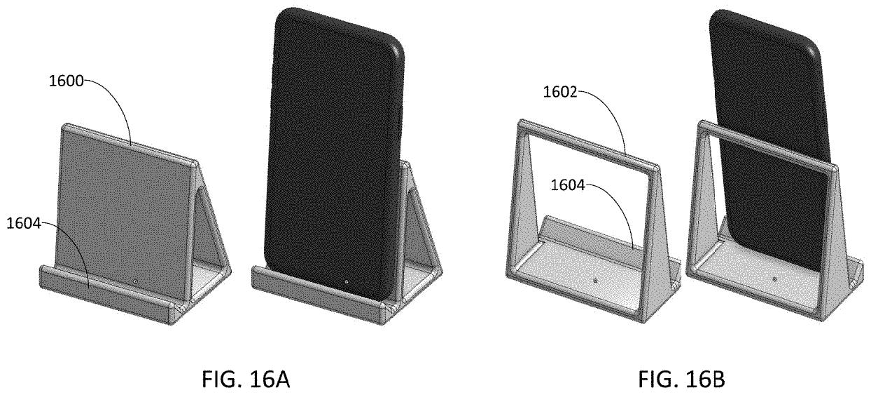

FIGS. 16A and 16B illustrate additional embodiments of device stands. In some embodiments, the device stands 1600 and 1602 can be manufactured through 3D printing techniques, although other manufacturing methods such as extrusion and/or injection molding are also possible. The stands 1600 and 1602 illustrated in FIGS. 16A and 16B may be a solid stand that is not foldable/unfoldable but rather, stays in a single shape and configuration. Relative to the stand 1600 illustrated in FIG. 16A, the stand 1602 of FIG. 16B has portions removed to reduce stand weight and material while retaining overall structure and function of the stand. Both stands 1600 and 1602 illustrated in FIGS. 16A and 16B include a ledge 1604 configured to support the base of a user device as it leans against a supporting portion of the stand. The user device may be placed such that the rearward-facing camera is oriented toward the supporting portion of the stand and the forward-facing camera is oriented toward the ledge 1604, or vice versa. In either configuration, both rearward- and forward-facing cameras can be uncovered such that full fields of view are accessible. While not shown, the stands 1600 and 1602 of FIGS. 16A and 16B may include a raised platform at their bases to elevate the user device. Various separate platforms of different heights may be included as part of a stand kit so that a user may customize the amount of elevation needed to achieve optimal positioning for their particular user device. The optimal platform may be removably or permanently fixed to the bottom of the stand.

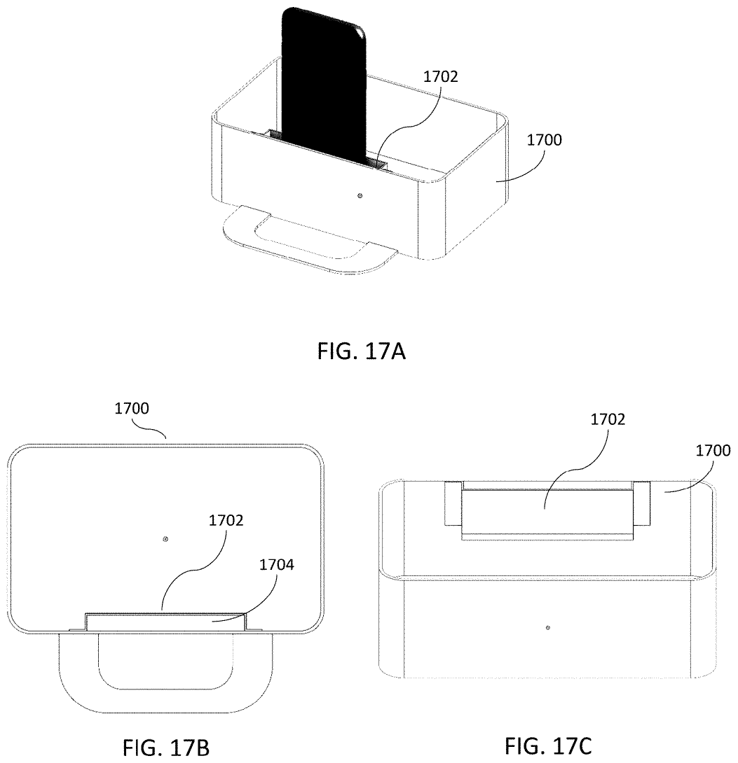

FIGS. 17A, 17B, and 17C illustrate another embodiment of a device stand. In this example, a piece of material 1702 is attached to the inside of a test kit box 1700 to act as a vertical stand. By attaching the piece of material 1702 to the inside of a side of the box, the piece of material 1702 and the box define a slot 1704 configured to receive and support the user device. While a vertical user device orientation is illustrated, the user device may alternatively be supported in a forward or backward leaning position depending on the size and configuration of the slot 1704, the piece of material 1702, and the box side. In some embodiments, as described above, a step may be included underneath the slot such that the user device is supported by the step when the user device is placed in the slot. This may provide elevation for the user device in order to achieve an acceptable view of the user and/or the test kit components from one or more cameras on the user device. In some embodiments, the material may comprise cardboard, plastic, metal, and/or biodegradable materials, although other materials are also possible. In some embodiments, the stand may be designed as part of one or more dividers, baffles, or other structures that are inserted in the box to organize the test components and/or to prevent the box from being crushed or damaged during shipping. In such embodiments, the piece of material may be attached to the divider, baffle, or other structure instead of or in addition to being attached to a side of the box. The dividers, baffles, or other structures may be integrally formed with the box or may be an additional inserted component. The dividers, baffles, or other structures coupled with the piece of material to form a slot may be movable relative to the box or may be in a fixed position. While FIGS. 17A-16C illustrate the piece of material being fixed to a long side of the box, it may alternatively or additionally be attached to a short side of a rectangular box. In some embodiments, more than one stand may be provided. Providing multiple slotted stands within the box may provide multiple options for the user to optimize their test setup and the views of user device cameras to include the user and the test kit components.

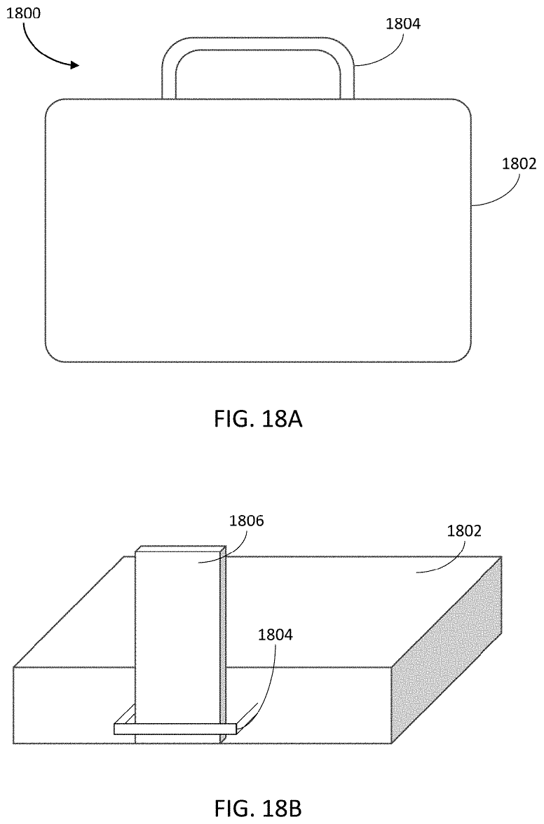

FIGS. 18A and 18B illustrates that, in some embodiments, the test kit box can include an integrated stand such that the user device 1806 is supported by an outside surface of the box. For example, as shown in FIGS. 18A and 18B, a test kit box 1800 can have a main body 1802 and a handle 1804. The handle 1804 of a test kit box 1800 can be configured as the stand. The handle 1804 may be located on an edge of the box 1800 such that when the box 1800 is placed on a surface, the handle 1804 rests on the surface. Alternatively, the handle 1804 may be positioned away from the edge of the box such that when the box 1800 is placed on a surface, the handle 1804 is elevated from the surface and provides additional support to prevent the user device 1806 tipping over. The handle 1804 may be coupled with a bottom portion of the box such that the lid can be opened and the handle 1804 remains stationary on the bottom portion of the box. This may allow for the user to open the box 1800 while the user device 1806 is positioned and may allow for one or more cameras on the user device 1806 to view the interior contents of the box. Specific sizes and shapes of the handle 1804 may be selected to support the user device 1806 in a desired position. For example, if a rearward lean is desired, the opening between the handle and the side of the box (i.e., the opening that is configured to receive the user device) may be larger such that the base of the user device can be positioned further away from the edge of the box and a top portion of the user device can lean against the edge of the box. In some embodiments, the box 1800 may include two handles to better support a variety of user device positions and to prevent the user device from tipping or falling over. Alternatively or additionally, handles may be constructed having a larger height dimension (e.g., a dimension out of the page in the illustration of FIG. 18A) to prevent the user device from tipping over and to provide a range of user device position options. In some embodiments, the opening between the handle and the side of the box should be sufficiently sized so that it can receive all manner of user devices.

Proper camera placement, for example as may be enabled by one or more of the embodiments described herein, can improve a user testing experience, but difficulties may still be encountered during a testing session. Accordingly, a test kit may be otherwise adapted to enable easy use by even novice users. In some embodiments, the test kit can be configured to facilitate monitoring or test result determination and/or interpretation by a remote proctor, a computer system, or both. It can be important for test materials to be placed and/or oriented properly so that the user, a remote proctor, or an automated computer system can monitor the testing session and determine if steps have been performed properly, if results are valid, and so forth. For example, invalid results can result from improper test-taking procedures (for example, not inserting a test swab sufficiently far into a solution, a user touching an active portion of a test strip, and so forth). Poor image quality (for example, lack of focus, poor lighting, poor color calibration, etc.) can make it difficult or even impossible for a remote proctor or computer system to accurately determine whether a testing procedure has been followed correctly and/or whether a result is positive, negative, or indeterminate.

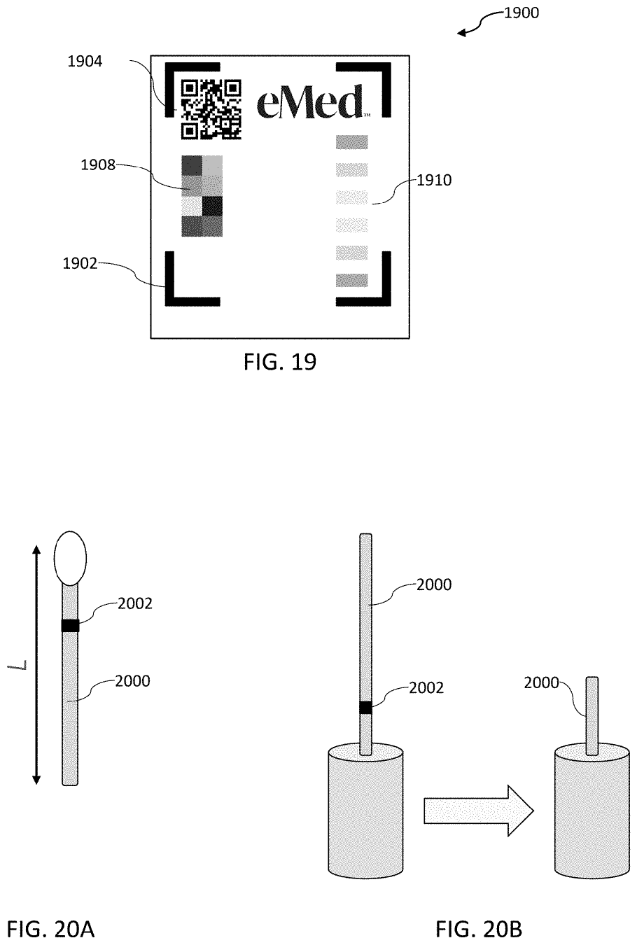

FIG. 19 illustrates embodiments of a reference card 1900 for a test kit and various features that can be included thereon. In some embodiments, test results can be interpreted by a proctor (e.g., a live person guiding the user through administration of the test using the user device) or by an artificial intelligence (AI) or computer vision (CV) system. In some embodiments, the test strip can be positioned in front of a reference card such that the reference card is a background for the sample when viewed by a user, a user device camera, a proctor, etc. which can include various features that facilitate interpretation of the test result.

The reference card may be a printed piece of cardstock, plastic, etc., with various elements and may be designed to include or exclude elements depending on test type or other factors. In some embodiments, the card may serve as background when taking an image of the test strip, so that all results images can be standardized. The card may provide a unique code 1904 (e.g., a QR Code) that can be quickly and reliably identified and scanned by a computer vision process. In some test methods, the code may be scanned before a user takes a test and after the user takes the test to ensure test continuity and security. The code may be referenced to a database containing lot number, expiration date, and other information specific to the user's test.

Various features that aid in obtaining an optimal camera image, from which results can be interpreted (e.g., by a trained guide or computer vision algorithm), may be included. For example, graduated color stripes 1910 (which may be, for example, of a constant hue and varying saturation and/or brightness) may be printed on the card, and an image that includes the stripes may be provided to a computer vision algorithm. If the algorithm can identify all shades of stripes, lightest through darkest, that image is considered to have sufficient quality for accurately detecting the result stripes. Similarly, color references 1908 may be included. The color references may be blocks or other printed areas that include known colors on which color calibration (e.g., white balance, contrast enhancement, etc.) may be based. In some embodiments, a system can be configured to extract graduated color stripes and/or color references from an image. In some embodiments, the system can recommend changes to lighting conditions, distance, etc., to improve image quality. In some embodiments, the system can adjust the color of the images using the extracted color references and known color references. This calibration step may assist results in performing accurate test result interpretation.

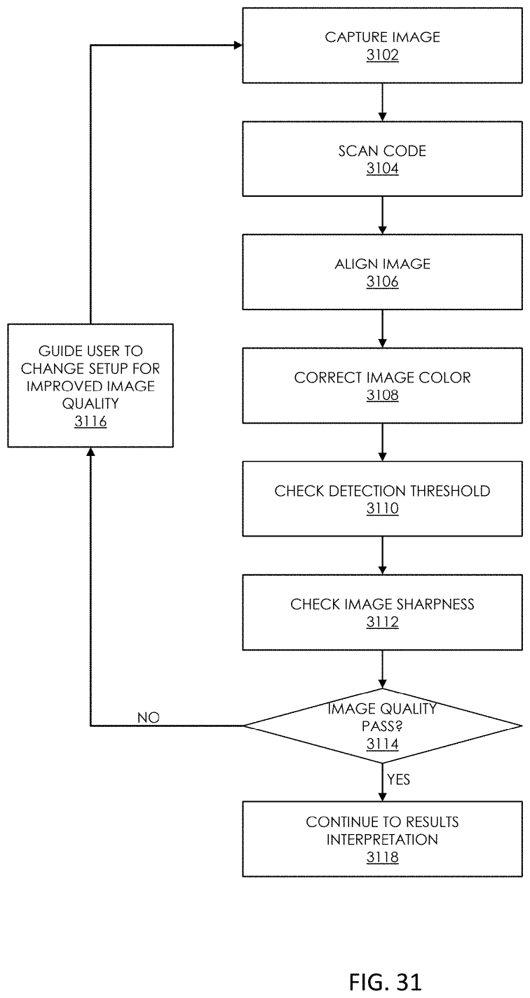

Various fiducials 1902 may be included on the card to aid in position calibration of the image and to provide an image post-processing algorithm with a basis upon which distortion correction may be performed (for example, removing a skew or keystone effect). One or more of the features described above may be used as part of an image pre-check before the user completes the testing process. The pre-check may use the features to determine whether adequate lighting is present, whether angle adjustments of the user device are needed, or whether other image quality adjustments are needed. If one or more of the pre-check items needs adjusting ahead of the test session, the image check algorithm or the test system may indicate to the user to make such adjustments (e.g., “please turn on a brighter light” or “please rotate the kit to face the nearest window”). An example method and process flow for such an image validation process is illustrated in FIG. 31.

FIG. 20A illustrates an example embodiment of a swab 2000 for a test kit and various features that can be included thereon. In some embodiments, a swab can have a length L, which can be, for example, approximately 3 inches in length or may be shorter or longer depending on the particular test and packaging constraints. The packaging box insert described above may include a slot or recess configured to hold the swab before and after use. Such a packaging feature may be particularly beneficial in keeping the swab away from possible contaminants that it would otherwise be exposed to if allowed to rest on a table or roll on the test surface. In an example, after use, at least a portion of the swab may be held within the sample tube to prevent spilling or falling. The swab may include an indicator 2002 such as a line or colored portion located at a specific distance from the sample collecting tip of the swab. The indicator 2002 may assist with computer vision or with proctor vision to ensure that the swab has been inserted at least a minimum distance during the sample collection phase. For example, as shown in FIG. 20B, the indicator 2002 may not be visible when the swab 2000 is inserted a sufficient minimum distance.

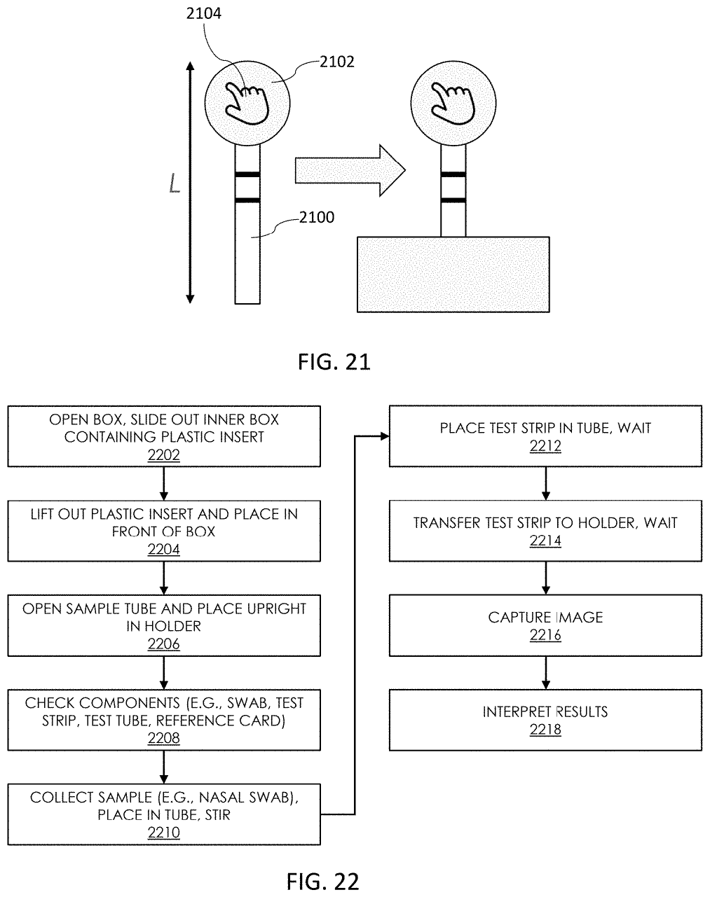

FIG. 21 illustrates an example embodiment of a test strip 2100 for a test kit and various features that can be included thereon. As shown in some embodiments, the test strip 2100 can include a grip 2102 positioned on one end that is configured to facilitate handling of the test strip. The grip 2102 may be formed from a material different from the chemically active test strip itself so as to prevent contamination or alteration of the sample testing process that could lead to inaccurate results. The grip 2102 can include an icon 2104 or other fiducial.

In some embodiments, the icon 2104 or other fiducial on the test strip (e.g., on the grip) can aid an AI or CV system in identifying the test strip within an image. The fiducial marker or other recognizable color or image may also be used in determining where the user is touching the test strip. For example, if the user's fingers are not positioned on the correct portion of the strip and are located on the chemically active test strip area, a proctor or other test security process may review the images to determine if the test is still valid. Further, FIG. 21 illustrates that, in some embodiments, the test strips can be configured such that the results region (e.g., the chemically active area of the sample test strip where stripes can appear) is visible when the test strip is inserted into a slot in an insert (for example, test strip slot 716 of the packaging 702). The test strip 2100 may be formed from a sufficiently rigid chemically active material such that the strip does not fall over when positioned in the slot. Alternatively, a structural support layer may be bonded to, or otherwise pressed against, the test strip 2100 or the slot may include a supporting surface to ensure the test strip does not bend or fold under its own weight during the testing procedure. In some embodiments, the test strip is approximately can have a length L which can be, for example, 3″ in length or may be longer or shorter depending on the particular test and/or packaging constraints.

FIG. 22 illustrates an embodiment of an example test flow or process. The illustrated steps are provided by way of example and should not be construed as limiting. In some embodiments, one or more steps can be omitted or altered. In some embodiments, one or more additional steps can be included. As shown in FIG. 22, a test flow can include, at 2202 opening a test box and removing the inner box containing a plastic insert. At 2204, a user can lift out the plastic insert and place it in front of the box. At 2206, the user can open a sample tube and place it upright in a holder. At 2208, a user can check the components of the text, such as the swab, test strip, test tube, and reference card. In some embodiments, a proctor, or an automated system (e.g., an AI system or CV system) can check the components. At 2210, the user can collect a sample (e.g., a nasal swab), place the swab in the test tube, and stir. At 2212, the user can place a test strip in the test tube and wait a prescribed period of time. In other embodiments, the user may add drops of solution from the test tube to the test strip instead of dipping the strip into the test tube. At 2214, the user can transfer the test strip to the holder and again wait for a prescribed period of time. At 2216, an image of the test strip can be captured. In some embodiments, the image can be captured by the users (for example, by tapping a button on the user's device. In other embodiments, the image can be captured automatically, for example after the prescribed amount of time has passed. At 2218, the results can be interpreted, for example by a proctor or by a machine using AI and/or CV.

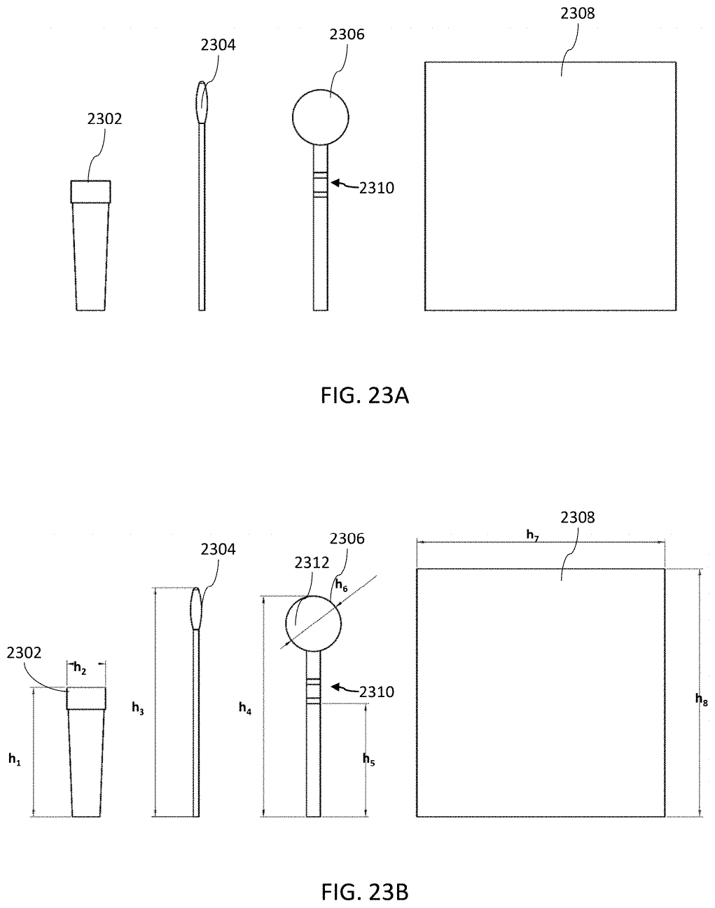

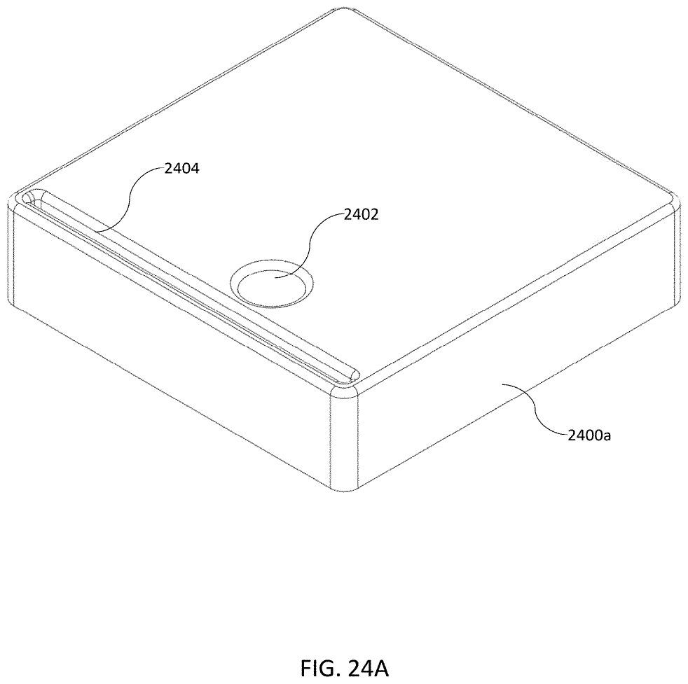

FIG. 23A illustrates embodiments of various components that can be included in some embodiments of a test kit. In the illustrated embodiment, the components include a sample tube 2302, a swab 2304, a test strip 2306 with a results region 2310, and a reference card 2308. In some embodiments, the reference card 2308 has at least one dimension (e.g., a height or a width) that is greater than the length of the test strip 2306. Such a relative size relationship may provide benefits when the reference card 2308 is positioned as a background behind the test strip 2306 within the insert (not shown). For example, the reference card 2308 having at least one dimension that is larger than the test strip length may allow for an image of the reference card and the test strip as captured by the user device to see the test strip clearly and fully because the reference card is located behind the entirety of the test strip. Additional relative size dimensions are illustrated in detail in FIG. 23B.

FIG. 23B illustrates example dimensional relationships between various components that can be included in some embodiments of a test kit according to some embodiments. The illustrated dimensional relationships need not be included in all embodiments and other dimensional relationships are possible.

As shown in FIG. 23B, in some embodiments h₆ of the test strip 2306 (e.g., the size of the grip) can be wider than h₂ of the sample tube 2302 (the size of the inlet to the sample tube). This can prevent the test strip 2306 from falling down into the sample tube 2302 and can also help to ensure that only the correct end of the test strip 2306 is inserted into the sample tube 2302. The test strip can include a grip 2312. While the grip 2312 is illustrated as a circle and the dimension h₆ is illustrated as a diameter of the circular grip 2312, other shapes and dimensions are possible without departing from the scope of the present application. For example, the grip 2312 may be a square, triangle, polygon, or other regular or irregular shape. The dimension h₆ may refer to the size dimension on an axis orthogonal to the long axis of the chemically active portion of the test strip 2306.

In some embodiments, the height h₃ of the swab 2304 and the height h₄ of the test strip 2306 can be less than the height h₆ of the reference card 2308. This can ensure that, when positioned in the testing configuration the swab and test strip are positioned in front of the reference card.

In some embodiments, the height h₅ of the results stripes region on the test strip is greater than the height h₁ of the sample tube. This can help to ensure that the results are visible to a user, a proctor, and/or a user device camera when the test strip is inserted into the sample tube.

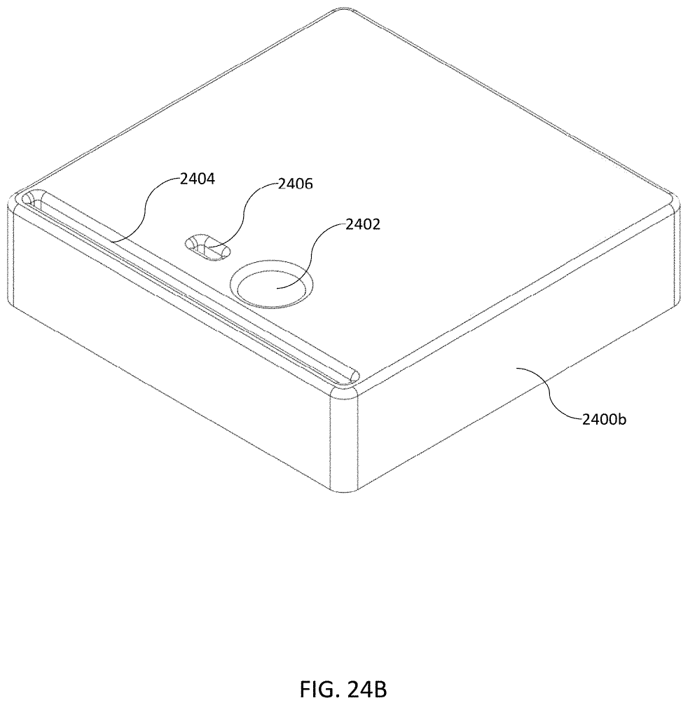

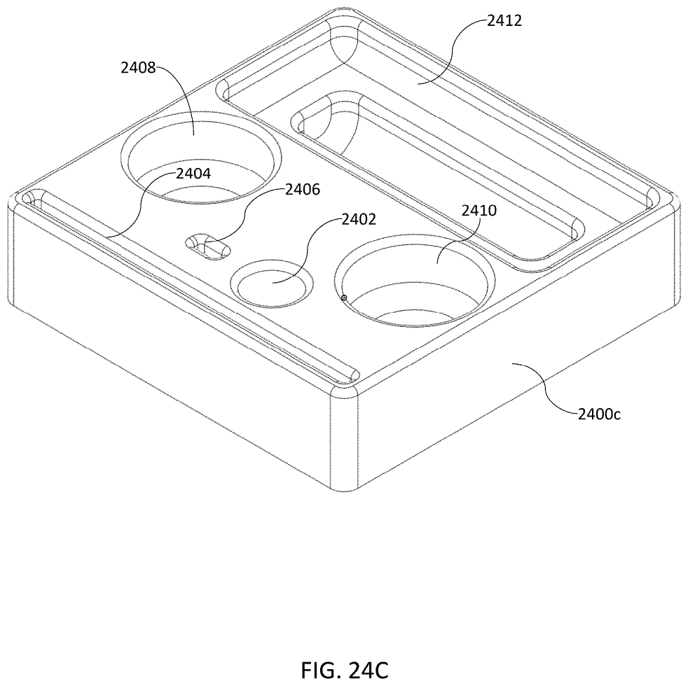

FIG. 24A illustrates an example insert for some embodiments of test kits. In this example, the insert/packing 2400a includes a recess 2402 for receiving the sample tube and a slot 2404 for receiving the reference card. While the reference card slot 2404 is located along an edge of the insert, other configurations are possible wherein the slot is farther away from the edge, wherein the slot is angled or non-parallel with respect to the edge of the insert, or wherein a base surface of the slot is parallel or non-parallel with a top plane of the insert packaging P.

FIG. 24B illustrates another example insert 2400b for some embodiments of test kits. In this example, the insert/packing 2400b includes a recess 2402 for receiving the sample tube, a slot 2404 for receiving the reference card, and a slot 2406 for receiving the test strip.

FIG. 24C illustrates another example insert 2400c for some embodiments of test kits. In this example, the insert/packing includes a recess 2402 for receiving the sample tube, a slot 2404 for receiving the reference card, and a slot 2406 for receiving the test strip. The packaging also includes features 2408, 2410, and 2412 formed therein. In some embodiments, features 2408 and 2410 are finger holes that facilitate lifting the insert. In some embodiments, 2412 is a recess that holds various components of the kit when in the packaged configuration.

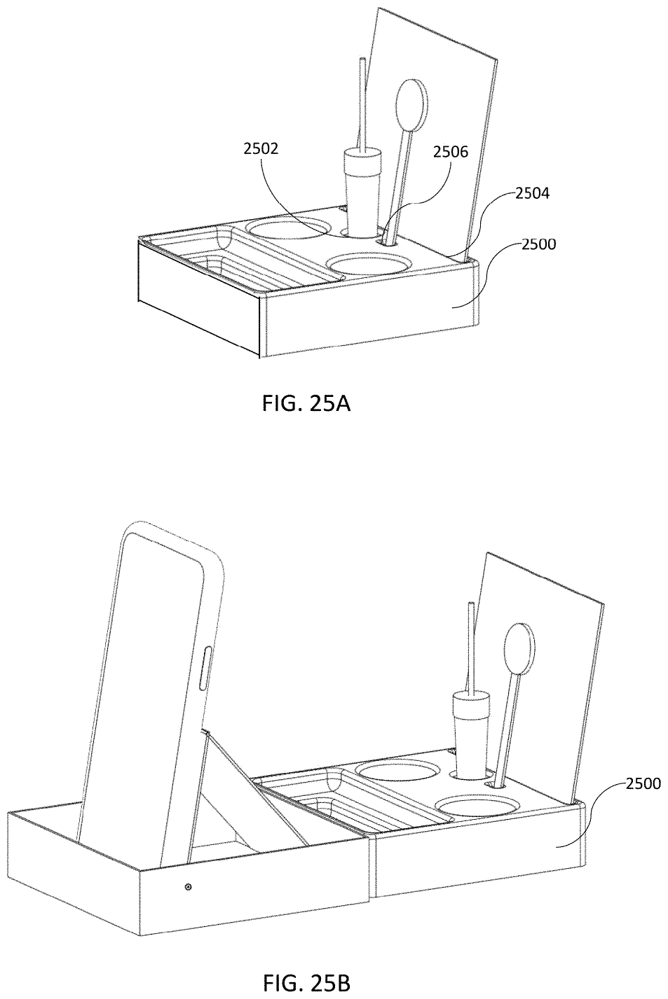

FIG. 25A illustrates an example insert for some embodiments of test kits with example components positioned therein in a testing configuration according to an embodiment. Within the packaging insert 2500, slot 2502 is configured to receive and hold a sample tube (e.g., sample tube 2302) in an upright position. The sample tube (e.g., sample tube 2302) may further include a test swab (e.g., test swab 2304) inserted therein, wherein the test swab has been used in collecting a sample from a user. The slot 2504 is configured to receive and hold a reference card (e.g., reference card 2308). Slot 2506 is configured to receive a test strip (e.g., test strip 2306) such that a results region (not shown) on the test strip is viewable relative to the reference card (e.g., reference card 2308) and is not covered by the insert or slot itself when viewed by a user, proctor, and/or a user device camera.

FIG. 25B illustrates an example insert for some embodiments of test kits with example components positioned therein and a user device in a stand of the test kit in a testing configuration according to an embodiment. Components and arrangements of the packaging insert 2500 may be similar to or the same as those illustrated in FIG. 25A.

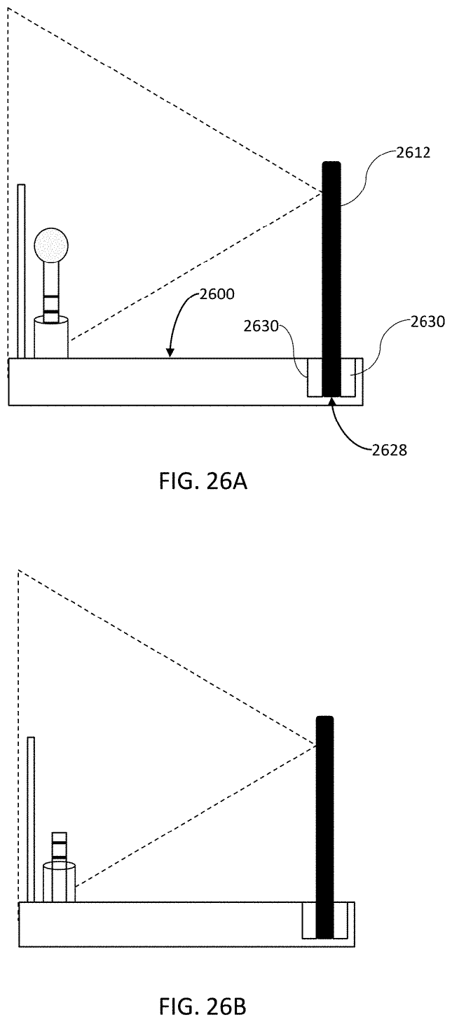

FIG. 26A illustrates embodiments of a test kit and various features that can be included therein. As shown, in this example, the packaging 2600 includes a slot 2628 configured to hold a user device 2612 (e.g., a smartphone). In some embodiments, the slot 2628 may include one or more foam (or other materials) inserts 2630 configured to stabilize the user device 2612 within the slot. The packaging may thus be configured to position the user device relative to the other components of the test kit in a testing configuration. The slot configuration may be used to hold the user device 2612 instead of or in addition to the foldable stands discussed with respect to previous embodiments. The user device slot 2628 may be parallel to a reference card slot (not shown) so as to position the reference card directly in front of and parallel to a focal plane associated with the user device camera. The sample tube slot and/or the test strip slot may be positioned between the reference card slot and the user device slot such that the reference card acts as a background for the test strip when the user device captures an image with a rearward-facing user device camera. The sample tube slot and/or the test strip slot may be located closer to the reference card slot than to the user device slot.

FIG. 26B illustrates another embodiment of a test kit and various features that can be included therein. The embodiment shown in FIG. 26A can be broadly similar to that shown in FIG. 26A and can differ in, for example, the relative placement of objects, characteristics of specific testing components (e.g., whether or not the test strip has a dedicated grip, and so forth).

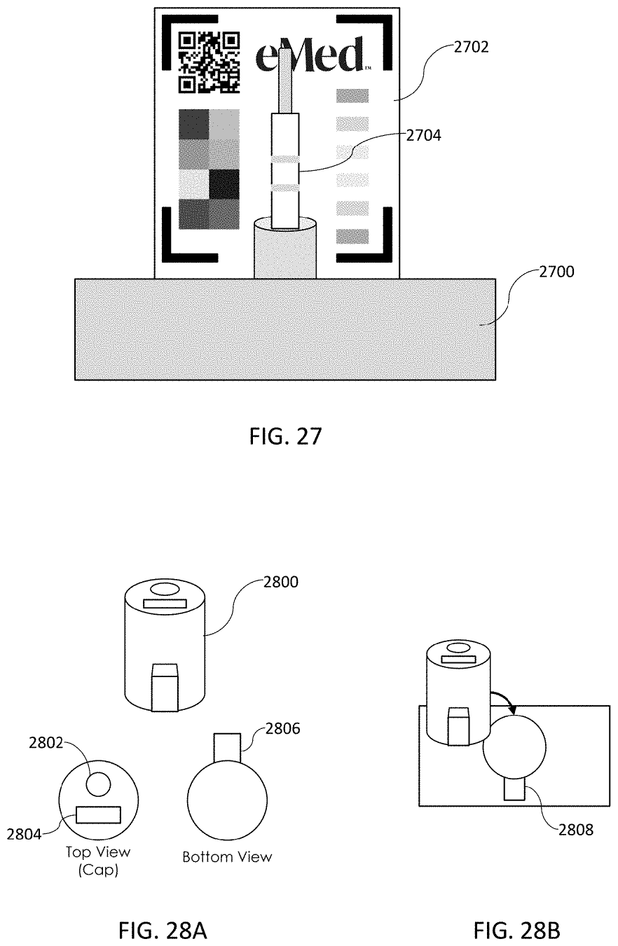

FIG. 27 illustrates an embodiment of a reference card 2702 for a test kit and various features that can be included thereon. In some embodiments, test results can be interpreted by a proctor (e.g., a live person guiding the user through administration of the test using the user device) or by an artificial intelligence (AI) or computer vision (CV) system. In some embodiments, the test strip 2704 can be positioned in front of a reference card which can include various features that facilitate interpretation of the test result. The reference card 2702 and test strip 2704 can be placed in test kit box 2700. The reference card 2702 can be similar to or the same as the reference card 1900 shown in FIG. 19. An example of a user device image quality check process is shown in FIG. 31.

FIGS. 28A and 28B illustrate embodiments of a sample tube 2800 for a test kit including various features that can be included thereon. In this example, the sample tube 2800 includes slots 2802 and 2804 formed therein for holding the swab and the test strip, respectively. In some embodiments, the sample tube 2800 may include a fluid (e.g., liquid) chemical, such as a buffer solution, to assist with completing the diagnostic test. When the swab and the test strip are both inserted into the sample tube 2800, the swab and the test strip may be exposed to the buffer solution, thereby combining the steps of exposing the swab sample to a buffer solution and exposing the test strip to the buffer solution and sample mixture.

In the illustrated example, the sample tube also includes a keying feature 2806 configured to correspond to a related feature 2808 in the packaging to specify the orientation of the sample tube 2800 relative to the packaging. This can be configured to ensure a desired orientation of the swab and test strip slots 2802 and 2804. For example, the sample tube 2800 may be keyed such that when inserted properly into the insert, the sample tube positions the test strip such that a results region of the test strip is viewable by a user device camera. In some embodiments, the plurality of slots provided in the sample tube may have different shapes. The shape of each slot may correspond to the particular test kit component that each slot is designed to receive. For example, the swab slot 2802 may be a circular slot to accommodate the circular cross-sectional shape and size of the swab while the test strip slot 2804 may be a rectangular slot to accommodate the rectangular cross-sectional shape and size of the test strip. Such a shape and size correlation between slot and test component may help a user to intuit which component should be placed in which slot to improve accuracy of the test process and to improve ease of use for the user.

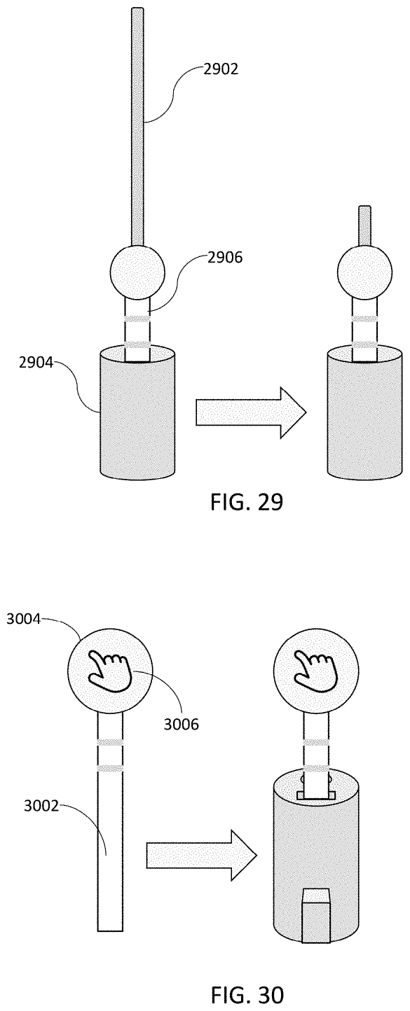

FIG. 29 illustrates embodiments of swabs for a test kit including various features that can be included thereon. In the examples of FIG. 29, the swab 2902 is shown inserted into the sample tube 2904 behind the test strip 2906. This configuration can be facilitated by the features described above with reference to FIGS. 28A and 28B.

FIG. 30 illustrates embodiments of test strips 3002 for a test kit including various features that can be included thereon. As shown, in some embodiments, the test strip 3002 can include a grip 3004 positioned on one end that is configured to facilitate handling of the test strip 3002. The grip 3004 can include an icon or other fiducial 3006. In some embodiments, a fiducial 3006 on the test strip (e.g., on the grip) can aid an AI or CV system in identifying the test strip within an image. Further, FIG. 30 illustrates that, in some embodiments, the test strip 3002 can be configured such that the results are visible when the test strip 3002 is inserted into the sample tube (e.g., the sample tube 2800). This can allow the results to be read without removing the test strip 3002 from the sample tube.

FIG. 31 is a block diagram that illustrates an example embodiment of a method of image preprocessing according to some embodiments. In some embodiments, the steps illustrated in FIG. 31 can be used for preprocessing an image before determining results. However, the steps of FIG. 31 can additionally or alternatively be used at other stages in the testing process, such as at the beginning of the process to determine if lighting conditions, camera quality, etc., are sufficient for carrying out the testing session. At 3102, the method can begin with an image of a reference card and a results strip being capture by a user device (e.g., a smartphone). At 3104, a computing system can recognize an identifier, such as a QR code, bar code, or other distinct code, on the reference card. In some embodiments, the code or unique identifier can be recognized using a computer vision algorithm. The QR code or other code can be a unique identifier for the test and can be different for each instance of a test that is manufactured. The identifier can be used to track the test throughout the testing process and to mitigate some types of fraud. In some embodiments, the code can include manufacturing information such as the lot number, which may be used to track manufacturing defects. In some embodiments, the identifier can include an expiration date that can be validated before testing. In some embodiments, the identifier can include a test type, version, etc., that can be looked up in a database to determine test strip interpretation information such as, for example, where various reference card features and/or test strip lines should appear, to determine a testing procedure, and so forth. In some embodiments, instead of or in addition to embedding some types of information in the identifier directly, the identifier can include information (e.g., a unique ID) that can be used to query an external source, such as an external database, to retrieve information such as the lot number, expiration date, test type, and so forth. In some embodiments, the identifier may not be unique. For example, the identifier may be used to identify a type of test, a manufacturing date range, a version of a test, and so forth, but may not be used to identify specific instances of a test.

At 3106, a computing system can be configured to align the image using one or more fiducials on the reference card. In some embodiments, the fiducials may be distinct features, while in other embodiments, the fiducials may be included in other features of the reference card, for example within the QR code. Aligning the image can include, for example, deskewing, removing keystoning, and so forth. At 3108, the system can perform color corrections to the image, for example using known reference colors printed onto the reference card. At 3110, the system may check to ensure that the user's device is capable of detecting a range of color shades (e.g., from light pink to dark pink) that are printed on the test card (for example, to ensure that the user's device can be used to detect a faint strip of color on a test strip). For example, a reference card can include a detection threshold calibration region having multiple color samples ranging from very light to relatively dark. At 3112, the system may check the sharpness of the image using a computer vision algorithm. After checking one or more of the above indicators of image quality, the system can decide if the image is of sufficient quality to be used in assisting with test result interpretation at 3114. If the image is not of sufficient quality, the system can, at 3116, prompt the user to take another photo with better lighting, less motion, no obstructions, etc. The new image can be processed through the same method to check for image quality. If the image passes the quality check, the image may continue on and be used in a results interpretation step 3118 where the test strip is compared to colors and/or shades printed on the reference card or to a reference that can be used to determine if a result is positive or negative.

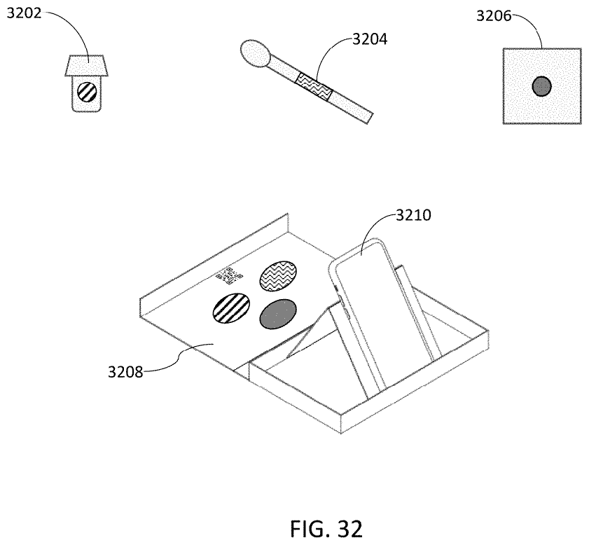

FIG. 32 illustrates that in some embodiments, one or more components of the test can be color coded, marked with shapes or symbols, and/or marked with patterns so that such items can be easily identifiable by the user, a proctor, and/or machine learning or computer vision algorithms. In some embodiments, the use of fiducials (whether color-based, pattern-based, shape-based, or otherwise) can facilitate automated assistance of proctoring functions, which can help to ensure that the user follows instructions successfully. For example, the user may be instructed to place the reagent cup 3202, marked with a circle containing angled lines, on a corresponding circle containing angled lines printed on the packaging 3208 of the test kit. This can help the user position the reagent cup 3202 within a field of view of the user device 3210. Similarly, the user can be instructed to swab his or her nose with the swab 3204 marked with wavy lines. The swab of the nose can be captured on the forward-facing camera of the user device 3210. After swabbing, the user can be instructed to place the swab 3204 on the circle with wavy lines on the packaging 3208, positioning the swab 3204 within the rearward-facing field of view of the user device 3210. The user can be instructed to place the test card 3206 in the corresponding area of the packaging 3208. While simple shapes filled with different patterns are described herein as a way to assist the user in positioning components of the test kit, other variations are possible. For example, outlines corresponding to the shapes of the components may be provided to show the user how to lay out the test kit components. These outlines may be color coded, patterned, etc., or may show only the shape of the corresponding components. The visual cues may also or alternatively include text labels (e.g., “reagent” or “swab”) or icon labels representative of the various components to indicate areas in which the test kit components should be placed.

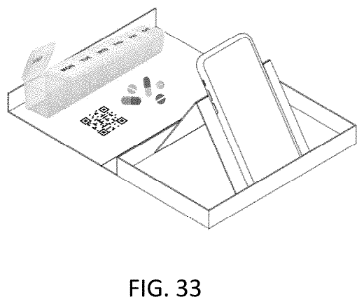

While the preceding disclosure provides examples related to medical testing procedures, the disclosures herein can also have other applications, such as aiding users in taking their prescription and/or non-prescription medications. FIG. 33 illustrates that mobile device stands can also be useful for medication monitoring. Some individuals may forget to take their medication, may not remember taking their medication, and so forth. Such problems may be exacerbated for individuals who take multiple medications, take medications at multiple times throughout the day, suffer from memory problems, and so forth. For example, a system can be provided for ensuring medication schedules are followed as prescribed. An application running on the user's phone can alert the user that it is time to take a medication. The user device (e.g., a smartphone) can be placed in a device stand, for example a stand as shown in FIG. 9 or other embodiments disclosed herein. A medication container may be placed within a field of view of the user device (e.g., a rearward-facing field of view) as illustrated. The medication container may be divided into multiple compartments (e.g., having one compartment per day of the week, two compartments per day of the week, etc.). The medication container may be provided to the user with the multiple compartments pre-filled with their prescription and/or non-prescription medication. Alternatively, the user may place medications received from their pharmacy into the correct compartments according to their prescribed medication schedule.

The application can instruct the user to open the appropriate compartment of the medication container (e.g., the Saturday morning compartment) and place the contents (e.g., pills, injections, syrups, etc.) in a designated area that is within a field of view of the camera. In some embodiments, a proctor or a computer vision algorithm can be configured to determine and verify which compartment was opened as well as the quantity, color, shape, lettering, or other markings (if present), and size of all pills or other medications in the area. In some embodiments, color and size can be determined using a printed fiducial/code next to the medication area as a reference. The determined type and quantity of medication present in the medication area can then be verified against the patient's medication schedule. Upon verification, the user can be instructed to take the medication. Otherwise, the user is alerted to any problems found (e.g., removal of wrong medication or the wrong quantities of medication). In some embodiments, a proctor or a computer vision algorithm can also be configured to verify that the user has properly ingested or otherwise taken the medication as instructed, for example using a forward-facing camera of the user device.

In some embodiments, the application can determine if there are known drug interactions. In some embodiments, the user can input information such as supplements, dietary practices, exercise, and so forth, and the application can use the user inputs to determine if there are possible interactions or other issues. In some embodiments, the application can be configured to provide warnings and/or notifications. For example, the application can provide a notification to the user when it is time to take a medication. In some embodiments, the application can alert a user than a dose has been missed. In some embodiments, the application can provide guidance such as reminding the user to take a medication with food, without food, at bedtime, after exercising, and so forth. In some embodiments, the application can be configured to provide warnings to the user, such as a warning to avoid taking a common over-the-counter medication that is known to have an adverse interaction with one or more of the user's prescription or non-prescription medications.

In some embodiments, the packaging (e.g., a lid of the box) could have the medication container built in such that the medication container is integrated, attached, or otherwise fixed to the packaging. As discussed above, the medication container (e.g., a pill dispenser) could be pre-filled with the user's prescription(s), and the box with pills included can be shipped to the user as a kit. In some embodiments, kits can be sent weekly or monthly. In some embodiments, the kits can comprise inexpensive materials, such as cardboard or plastic, and can be recyclable and/or compostable after use. In addition to pills, test kits can be configured to monitor other medical parameters, such as glucose monitoring.

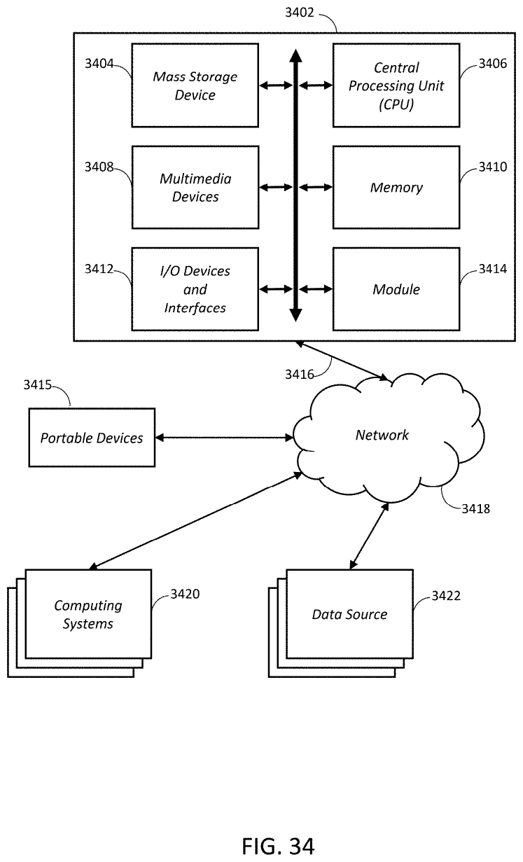

FIG. 34 is a block diagram depicting an embodiment of a computer hardware system configured to run software for implementing one or more embodiments of the health testing and diagnostic systems, methods, and devices disclosed herein.

In some embodiments, the systems, processes, and methods described herein are implemented using a computing system, such as the one illustrated in FIG. 34. The example computer system 3402 is in communication with one or more computing systems 3420 and/or one or more data sources 3422 via one or more networks 3418. While FIG. 34 illustrates an embodiment of a computing system 3402, it is recognized that the functionality provided for in the components and modules of computer system 3402 may be combined into fewer components and modules, or further separated into additional components and modules.

The computer system 3402 can comprise a health testing and diagnostic module 3414 that carries out the functions, methods, acts, and/or processes described herein. The health testing and diagnostic module 3414 is executed on the computer system 3402 by a central processing unit 3406 discussed further below.

In general, the word “module,” as used herein, refers to logic embodied in hardware or firmware or to a collection of software instructions, having entry and exit points. Modules are written in a program language, such as JAVA, C or C++, PYTHON, or the like. Software modules may be compiled or linked into an executable program, installed in a dynamic link library, or may be written in an interpreted language such as BASIC, PERL, LUA, or Python. Software modules may be called from other modules or from themselves, and/or may be invoked in response to detected events or interruptions. Modules implemented in hardware include connected logic units such as gates and flip-flops, and/or may include programmable units, such as programmable gate arrays or processors.

Generally, the modules described herein refer to logical modules that may be combined with other modules or divided into sub-modules despite their physical organization or storage. The modules are executed by one or more computing systems and may be stored on or within any suitable computer readable medium or implemented in-whole or in

part within special designed hardware or firmware. Not all calculations, analysis, and/or optimization require the use of computer systems, though any of the above-described methods, calculations, processes, or analyses may be facilitated through the use of computers. Further, in some embodiments, process blocks described herein may be altered, rearranged, combined, and/or omitted.

The computer system 3402 includes one or more processing units (CPU) 3406, which may comprise a microprocessor. The computer system 3402 further includes a physical memory 3410, such as random-access memory (RAM) for temporary storage of information, a read only memory (ROM) for permanent storage of information, and a mass storage device 3404, such as a backing store, hard drive, rotating magnetic disks, solid state disks (SSD), flash memory, phase-change memory (PCM), 3D XPoint memory, diskette, or optical media storage device. Alternatively, the mass storage device may be implemented in an array of servers. Typically, the components of the computer system 3402 are connected to the computer using a standards-based bus system. The bus system can be implemented using various protocols, such as Peripheral Component Interconnect (PCI), Micro Channel, SCSI, Industrial Standard Architecture (ISA) and Extended ISA (EISA) architectures.

The computer system 3402 includes one or more input/output (I/O) devices and interfaces 3412, such as a keyboard, mouse, touch pad, and printer. The I/O devices and interfaces 3412 can include one or more display devices, such as a monitor, that allows the visual presentation of data to a user. More particularly, a display device provides for the presentation of GUIs as application software data, and multi-media presentations, for example. The I/O devices and interfaces 3412 can also provide a communications interface to various external devices. The computer system 3402 may comprise one or more multi-media devices 3408, such as speakers, video cards, graphics accelerators, and microphones, for example.

The computer system 3402 may run on a variety of computing devices, such as a server, a Windows server, a Structure Query Language server, a Unix Server, a personal computer, a laptop computer, and so forth. In other embodiments, the computer system 3402 may run on a cluster computer system, a mainframe computer system and/or other computing system suitable for controlling and/or communicating with large databases, performing high volume transaction processing, and generating reports from large databases. The computing system 3402 is generally controlled and coordinated by an operating system software, such as z/OS, Windows, Linux, UNIX, BSD, SunOS, Solaris, MacOS, or other compatible operating systems, including proprietary operating systems. Operating systems control and schedule computer processes for execution, perform memory management, provide file system, networking, and I/O services, and provide a user interface, such as a graphical user interface (GUI), among other things.

The computer system 3402 illustrated in FIG. 34 is coupled to a network 3418, such as a LAN, WAN, or the Internet via a communication link 3416 (wired, wireless, or a combination thereof). Network 3418 communicates with various computing devices and/or other electronic devices. Network 3418 is communicating with one or more computing systems 3420 and one or more data sources 3422. The health testing and diagnostic module 3414 may access or may be accessed by computing systems 3420 and/or data sources 3422 through a web-enabled user access point. Connections may be a direct physical connection, a virtual connection, and other connection type. The web-enabled user access point may comprise a browser module that uses text, graphics, audio, video, and other media to present data and to allow interaction with data via the network 3418.

Access to the health testing and diagnostic module 3414 of the computer system 3402 by computing systems 3420 and/or by data sources 3422 may be through a web enabled user access point such as the computing systems' 3420 or data source's 3422 personal computer, cellular phone, smartphone, laptop, tablet computer, e-reader device, audio player, or another device capable of connecting to the network 3418. Such a device may have a browser module that is implemented as a module that uses text, graphics, audio, video, and other media to present data and to allow interaction with data via the network 3418.

The output module may be implemented as a combination of an all-points addressable display such as a cathode ray tube (CRT), a liquid crystal display (LCD), a plasma display, or other types and/or combinations of displays. The output module may be implemented to communicate with input devices 3412 and they also include software with the appropriate interfaces which allow a user to access data through the use of stylized screen elements, such as menus, windows, dialogue boxes, tool bars, and controls (for example, radio buttons, check boxes, sliding scales, and so forth). Furthermore, the output module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition, a touch screen may act as a hybrid input/output device. In another embodiment, a user may interact with the system more directly such as through a system terminal connected to the score generator without communications over the Internet, a WAN, or LAN, or similar network.

In some embodiments, the system 3402 may comprise a physical or logical connection established between a remote microprocessor and a mainframe host computer for the express purpose of uploading, downloading, or viewing interactive data and databases online in real time. The remote microprocessor may be operated by an entity operating the computer system 3402, including the client server systems or the main server system, an/or may be operated by one or more of the data sources 3422 and/or one or more of the computing systems 3420. In some embodiments, terminal emulation software may be used on the microprocessor for participating in the micro-mainframe link.

In some embodiments, computing systems 3420 who are internal to an entity operating the computer system 3402 may access the health testing and diagnostic module 3414 internally as an application or process run by the CPU 3406.

In some embodiments, one or more features of the systems, methods, and devices described herein can utilize a URL and/or cookies, for example for storing and/or transmitting data or user information. A Uniform Resource Locator (URL) can include a web address and/or a reference to a web resource that is stored on a database and/or a server. The URL can specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Domain Name System (DNS) can look up the URL and its corresponding IP address. URLs can be references to web pages, file transfers, emails, database accesses, and other applications. The URLs can include a sequence of characters that identify a path, domain name, a file extension, a host name, a query, a fragment, scheme, a protocol identifier, a port number, a username, a password, a flag, an object, a resource name and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user's computer. This data can be stored by a user's web browser while the user is browsing. The cookies can include useful information for websites to remember prior browsing information, such as a shopping cart on an online store, clicking of buttons, login information, and/or record of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a web site). The cookie data can be encrypted to provide security for the consumer. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

The computing system 3402 may include one or more internal and/or external data sources (for example, data sources 3422). In some embodiments, one or more of the data repositories and the data sources described above may be implemented using a relational database, such as DB2, Sybase, Oracle, CodeBase, and Microsoft® SQL Server as well as other types of databases such as a flat-file database, an entity relationship database, and object-oriented database, and/or a record-based database.

The computer system 3402 may also access one or more databases 3422. The databases 3422 may be stored in a database or data repository. The computer system 3402 may access the one or more databases 3422 through a network 3418 or may directly access the database or data repository through I/O devices and interfaces 3412. The data repository storing the one or more databases 3422 may reside within the computer system 3402.

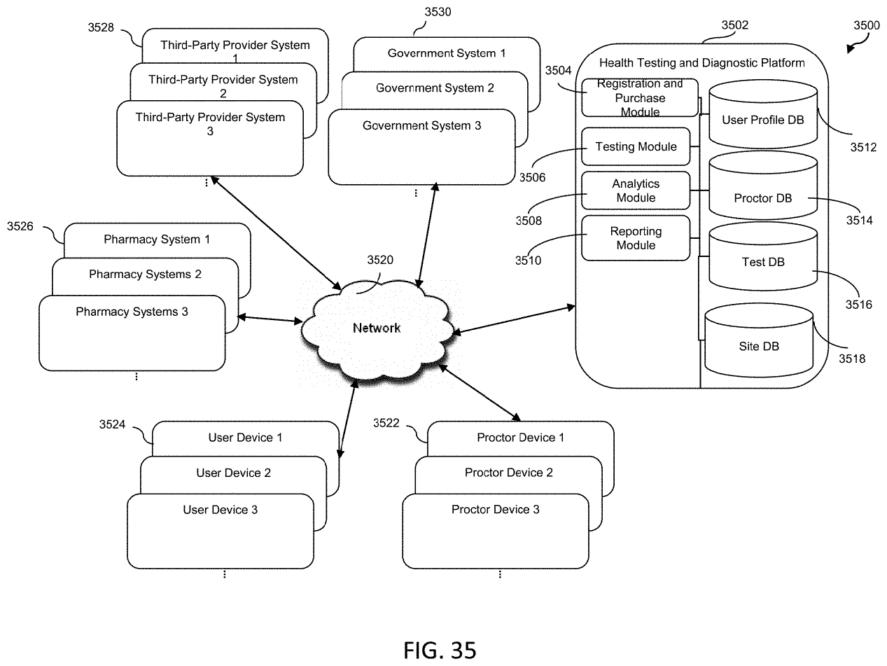

FIG. 35 is a block diagram illustrating an example embodiment of a computer system 3500 configured to run software for implementing one or more embodiments of the health testing and diagnostic systems, methods, and devices disclosed herein. In some embodiments, the various systems, methods, and devices described herein may also be implemented in decentralized systems such as, for example, blockchain applications. For example, blockchain technology may be used to maintain user profiles, proctor profiles, test results, test site databases, and/or financing databases or ledgers, dynamically generate, execute, and record testing plan agreements, perform searches, conduct patient-proctor matching, determine pricing, and conduct any other functionalities described herein.

In some embodiments, a health testing and diagnostic platform 3502 may be comprised of a registration and purchase module 3504, a testing module 3506, an analytics module 3508, and a reporting module 3510. The health testing and diagnostic platform 3502 may also comprise a user profile database 3512, a proctor database 3514, a test database 3516, and/or a site database 3518. The health testing and diagnostic platform 3502 can be connected to a network 3520. The network 3520 can be configured to connect the health testing and diagnostic platform 3502 to one or more proctor devices 3522, one or more user devices 3524, one or more pharmacy systems 3526, one or more third-party provider systems 3528, and/or one or more government systems 3530.

The registration and purchase 3504 may function by facilitating patient registration through one or more registration interfaces and in conjunction with the user database 3512, store user registration data. The testing module 3506 may be configured to allow a user to initiate and complete a medical test or visit with a proctor through a series of pre-testing and testing interfaces, as described herein. The analytics module 3508 may be configured to dynamically analyze patient tests across a given population stored in the test database 3516 and provide structured data of the test results. The reporting module 3510 may function by dynamically and automatically reporting test results to government entities, patients, and third parties using one or more interfaces, such as one or more application programming interfaces. Each of the modules can be configured to interact with each other and the databases discussed herein.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than restrictive sense.

Indeed, although this invention has been disclosed in the context of certain embodiments and examples, it will be understood by those skilled in the art that the invention extends beyond the specifically disclosed embodiments to other alternative embodiments and/or uses of the invention and obvious modifications and equivalents thereof. In addition, while several variations of the embodiments of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes of the embodiments of the disclosed invention. Any methods disclosed herein need not be performed in the order recited. Thus, it is intended that the scope of the invention herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and subcombinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination. No single feature or group of features is necessary or indispensable to each and every embodiment.

It will also be appreciated that conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

Further, while the methods and devices described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (e.g., as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the embodiments shown herein, but are to be accorded the widest scope consistent with this disclosure, the principles and the novel features disclosed herein.

What is claimed is:

1. A method for remote diagnostic testing comprising:
receiving, by a computer system from a user device, a first image frame;
determining, by the computer system, a first area of the first image frame;
identifying, by the computer system, a first feature in the first area;
providing, by the computer system to a user via the user device, an indication that the first feature is in the first area of the first image frame; and
continuing, by the computer system, a remote diagnostic testing session.

2. The method of claim 1, further comprising:
receiving, by the computer system from the user device, a second image frame;
identifying, by the computer system, a second area of the second image frame;
determining, by the computer system, that the first feature is not within the second area of the second image frame;
providing, by the computer system to the user via the user device, an indication that the first feature is not in the second area of the second image frame; and
pausing, by the computer system, the remote diagnostic testing session.

3. The method of claim 2, further comprising:
receiving, by the computer system form the user device, a third image frame;
identifying, by the computer system, a third area of the third image frame;
determining, by the computer system, that the first feature is within the third area of the third image frame;
providing, by the computer system to the user via the user device, an indication that the first feature is within the third area of the third image frame; and
resuming, by the computer system, the remote diagnostic testing session.

4. The method of claim 3, wherein the second area is the same as the first area and the third area is the same as the second area.

5. The method of claim 1, wherein the first feature comprises a test kit, a swab, a test strip, a reagent bottle, or a reference card.

6. The method of claim 5, wherein the first feature comprises a reference card.

7. The method of claim 6, wherein the reference card comprises a unique identifier of a test.

8. The method of claim 6, further comprising:
detecting, by the computer system, one or more fiducials in the first image frame; and
adjusting an alignment of the first image frame, wherein adjusted the alignment comprises adjust one or more of skew and keystone.

9. The method of claim 6, further comprising:
identifying, by the computer system, a detection threshold calibration area;
determining, by the computer system, a first color of a first region of the detection threshold calibration area;
determining, by the computer system, a second color of a second region of the detection threshold calibration area;
determining, by the computer system, if a difference between the first color and the second color is greater than or equal to a minimum difference value; and
if the difference is greater than or equal to the minimum difference value, continuing, by the computer system, the remote diagnostic testing session;
otherwise, providing, by the computer system via the user device, an indication to the user that the difference is less than the minimum difference value.

10. The method of claim 6, further comprising:
identifying, by the computer system, a color calibration area;
extracting, by the computer system, a first color value from a first portion of the color calibration area; and
adjusting, by the computer system, the first image frame based on a difference between the first color value and a first reference color value.

11. The method of claim 10, further comprising:
extracting, by the computer system, a second color value from a second portion of the color calibration area; and
adjusting, by the computer system, the first image frame based on a difference between the second color value and a second reference color value.

12. The method of claim 7, further comprising:
determining, by the computer system based on the unique identifier, an expiration date of a test kit associated with the reference card; and
validating, by the computer system, that the test kit is not expired.

13. The method of claim 7, further comprising:
querying, by the computer system based on the unique identifier, a database; and
receiving, by the computer system from the database, information about the test, the information comprising one or more of reference card feature locations, test strip interpretation information, test strip line locations, and testing procedures.

14. The method of claim 1, further comprising:
determining, by the computer system, a sharpness of the first image frame.

15. A system for remote diagnostic testing comprising:
a non-transitory computer-readable medium with instructions encoded thereon; and
one or more processors configured to execute the instructions to cause the system to:
receive, by a computer system from a user device, a first image frame;
determine a first area of the first image frame;
identify a first feature in the first area;
provide, to a user via the user device, an indication that the first feature is in the first area of the first image frame; and
continue a remote diagnostic testing session.

16. The system of claim 15, wherein the instructions, when executed by the one or more processors, further cause the system to:
receive, by the computer system from the user device, a second image frame;
identify a second area of the second image frame;
determine that the first feature is not within the second area of the second image frame;
provide, to the user via the user device, an indication that the first feature is not in the second area of the second image frame; and
pause the remote diagnostic testing session.

17. The system of claim 16, wherein the instructions, when executed by the one or more processors, further cause the system to:
receive, by the computer system form the user device, a third image frame;
identify a third area of the third image frame;
determine that the first feature is within the third area of the third image frame;
provide, to the user via the user device, an indication that the first feature is within the third area of the third image frame; and
resume the remote diagnostic testing session.

18. The system of claim 17, wherein the second area is the same as the first area and the third area is the same as the second area.

19. The system of claim 15, wherein the first feature comprises a test kit, a swab, a test strip, a reagent bottle, or a reference card.

20. The system of claim 19, wherein the first feature comprises a reference card.

21. The system of claim 20, wherein the reference card comprises a unique identifier of a test.

22. The system of claim 20, wherein the instructions, when executed by the one or more processors, further cause the system to:
detect one or more fiducials in the first image frame; and
adjust an alignment of the first image frame, wherein adjusted the alignment comprises adjust one or more of skew and keystone.

23. The system of claim 20, wherein the instructions, when executed by the one or more processors, further cause the system to:
identify a detection threshold calibration area;
determine a first color of a first region of the detection threshold calibration area;
determine a second color of a second region of the detection threshold calibration area;
determine if a difference between the first color and the second color is greater than or equal to a minimum difference value; and
if the difference is greater than or equal to the minimum difference value, continue the remote diagnostic testing session;
otherwise, provide, via the user device, an indication to the user that the difference is less than the minimum difference value.

24. The system of claim 20, wherein the instructions, when executed by the one or more processors, further cause the system to:
identify a color calibration area;
extract a first color value from a first portion of the color calibration area; and
adjust the first image frame based on a difference between the first color value and a first reference color value.

25. The system of claim 24, wherein the instructions, when executed by the one or more processors, further cause the system to:
extract a second color value from a second portion of the color calibration area; and
adjust the first image frame based on the difference between the second color value and a second reference color value.

26. The system of claim 21, wherein the instructions, when executed by the one or more processors, further cause the system to:
determine, by the computer system based on the unique identifier, an expiration date of a test kit associated with the reference card; and
validate that the test kit is not expired.

27. The system of claim 21, wherein the instructions, when executed by the one or more processors, further cause the system to:
query, by the computer system based on the unique identifier, a database; and
receive, by the computer system from the database, information about the test, the information comprising one or more of reference card feature locations, test strip interpretation information, test strip line locations, and testing procedures.

28. The system of claim 15 wherein the instructions, when executed by the one or more processors, further cause the system to determine a sharpness of the first image frame.
